SEQUENCE LISTING (17 sequences)

SEQ ID NO: 1 - protein, 696 aa, Oocystis sp.
MAMPINPYNAKHETAVVWFRRDLRISDNPALAAALQSATNVIPLFIWAPEEEGQFQPGRANRWWLRNSLLSLSAELERLGSRLVCMAAPDCQSALCHVLASTGASALFTNRLYDPITMVRDNELLARVAGLGVRCFMFNSDLLYEPWEVLDQAGKPFNSFDAFWGRSPLPFSLEHAHRVTHMPHPPLPPIPAPVALPTVPGSVCGTLVDSLGLLTEEELLTNQQLEFTWSPGSAGAHKLFAKFVSRRLRQFQHDRAKSDRNSTSRLSPHVHFGEISVRYMYYVVKQKEAEWLATGERITSCADFLQQMGYREYSRYLSWHFPFTHERSLLEHVRACPYRMDQRLFKAWRQGNTGYPLVDAAMRELWSSGWMHNRMRVTAASFLVKHLLLPWQWGLKHFWDALVDADLEADALGWQYCAGCLADAHPLDYMIDHGTESKRFDPDGQYVRRWLPVLARVPRQYIHEPWTAPQHVLDEAGVELGSNYPWPVVDAGEAQVLLGAAKDVVDAALEALNETQRGPYRPPSLNPLDPSAPDSLRRMYAKFQHQLTRTAESAASGQRPSKRTTMEAAAQQAREDMQVCGQGQGGSSDNEEVESNMGVVDALLGGMAQLAGDDTAEVVQRRHVHRRRPSSCATGGSTQDGPLARRPAAGSGCGSAPDAPQQDGASPDAPWQHEAPPRGNGQAKHAALQPSNAWGS

SEQ ID NO: 2 - DNA, 2091 bp, Oocystis sp.
  source 1..2091: mol_type = other DNA
atggcgatgccgataaacccgtacaatgcaaagcacgagacagcggttgtatggttcaggcgggacttgcgcatctctgataacccggctctagccgcagcgctccagtcggcgaccaacgtgatcccgctgttcatatgggcgccagaggaggagggccagttccagccgggccgtgctaaccgatggtggctgcgcaactcgctgctgtcgctgtccgccgagctcgagcggctgggcagcaggctggtgtgcatggccgcgcccgactgccagtcggcgctctgccacgtgctggccagcaccggtgccagcgcgctgttcaccaaccgactgtacgatcccatcaccatggtgagggacaacgagctgctcgcacgcgtggccggcctgggtgtgcgctgcttcatgtttaactccgacctgctgtacgagccatgggaggtgctggaccaggcgggcaagccgttcaactcgttcgacgcgttttggggcaggtcccctttgcccttctcactggaacacgcgcacagggtgacgcacatgccgcacccgcccctgcctccgatcccggcgcccgtggcgctccccaccgtgcctggcagcgtgtgcggcacgctggtcgactcccttggcctactgacggaggaggagctgctgactaaccagcagctggagttcacgtggtccccgggcagtgccggtgcgcacaagctgtttgccaagtttgtgtccaggcgtcttcgccagttccagcacgatcgggccaagtcagaccggaacagcacctcgcgcctgtcgccgcacgtgcacttcggcgagatcagcgtgcggtacatgtactacgtggtcaagcagaaggaagccgagtggctggcgacgggagagcgcatcaccagctgtgcggacttcctgcagcagatggggtaccgcgagtactcgcgctacctctcctggcacttccccttcacccacgagcgctccctgcttgagcacgtgcgcgcctgcccgtaccgcatggaccaaaggctgttcaaggcttggcggcagggcaacaccgggtaccctctggttgacgcggccatgcgagagctgtggagctccggctggatgcacaaccgcatgagggtgaccgctgcttcctttttggtcaagcacctgctgctgccctggcagtggggcctgaagcacttttgggacgcgctggtggacgcagatctggaggccgatgcactggggtggcagtactgcgcaggctgcctggcagacgcacaccccctggactacatgatcgaccacgggaccgagtccaagcggtttgaccccgacggccagtacgtgcgccgctggctgcccgtgctggcaagggtgcccaggcaatacatacacgagccgtggaccgcaccgcagcatgtgctggacgaggcgggcgtcgagctcggctccaactacccctggccggtggtcgacgcgggggaggcgcaggtgctgctgggcgctgccaaggacgtggtggacgccgccctggaggcgctcaacgagacgcagagagggccctaccggccgcccagcctcaaccccctcgacccatccgcgccggactcgctgcggcgcatgtatgccaagttccagcaccagctcacgcgcacggcggagtcggcggccagcgggcagcggccctccaagcggacaacgatggaggcagcagcgcagcaggcacgggaggacatgcaggtgtgcggccagggccagggcggctcctctgacaacgaggaagtcgagagcaacatgggggtggttgacgcgctgctcggcggcatggcgcaactcgccggtgatgacacggcggaggtggtgcagcgccgccacgtgcaccgccgccggccgtcctcctgcgcaacaggaggcagtacgcaggacggcccgctcgcgcgccgacccgccgccggcagcggatgcggcagcgcgccagatgctccacagcaagatggggcatcgccagacgctccttggcaacacgaggcaccgccccgaggcaatggccaagcaaagcatgcggctctgcagccaagcaacgcatggggctcttga

SEQ ID NO: 3 - protein, 55 aa, synthetic construct
MAMPINPYNAKHETAVVWFRRDLRISDNPALAAALQSATNVIPLFIWAPEEEGQF

SEQ ID NO: 4 - DNA, 2091 bp, synthetic construct
  source 1..2091: mol_type = other DNA
atggcgatgccgataaacccgtacaatgcaaagcacgagacagcggttgtatggttcaggcgggacttgcgcatctctgataacccggctctagccgcagcgctccagtcggcgaccaacgtgatcccgctgttcatatgggcgccagaggaggagggccagttctagccgggccgtgctaaccgatggtggctgcgcaactcgctgctgtcgctgtccgccgagctcgagcggctgggcagcaggctggtgtgcatggccgcgcccgactgccagtcggcgctctgccacgtgctggccagcaccggtgccagcgcgctgttcaccaaccgactgtacgatcccatcaccatggtgagggacaacgagctgctcgcacgcgtggccggcctgggtgtgcgctgcttcatgtttaactccgacctgctgtacgagccatgggaggtgctggaccaggcgggcaagccgttcaactcgttcgacgcgttttggggcaggtcccctttgcccttctcactggaacacgcgcacagggtgacgcacatgccgcacccgcccctgcctccgatcccggcgcccgtggcgctccccaccgtgcctggcagcgtgtgcggcacgctggtcgactcccttggcctactgacggaggaggagctgctgactaaccagcagctggagttcacgtggtccccgggcagtgccggtgcgcacaagctgtttgccaagtttgtgtccaggcgtcttcgccagttccagcacgatcgggccaagtcagaccggaacagcacctcgcgcctgtcgccgcacgtgcacttcggcgagatcagcgtgcggtacatgtactacgtggtcaagcagaaggaagccgagtggctggcgacgggagagcgcatcaccagctgtgcggacttcctgcagcagatggggtaccgcgagtactcgcgctacctctcctggcacttccccttcacccacgagcgctccctgcttgagcacgtgcgcgcctgcccgtaccgcatggaccaaaggctgttcaaggcttggcggcagggcaacaccgggtaccctctggttgacgcggccatgcgagagctgtggagctccggctggatgcacaaccgcatgagggtgaccgctgcttcctttttggtcaagcacctgctgctgccctggcagtggggcctgaagcacttttgggacgcgctggtggacgcagatctggaggccgatgcactggggtggcagtactgcgcaggctgcctggcagacgcacaccccctggactacatgatcgaccacgggaccgagtccaagcggtttgaccccgacggccagtacgtgcgccgctggctgcccgtgctggcaagggtgcccaggcaatacatacacgagccgtggaccgcaccgcagcatgtgctggacgaggcgggcgtcgagctcggctccaactacccctggccggtggtcgacgcgggggaggcgcaggtgctgctgggcgctgccaaggacgtggtggacgccgccctggaggcgctcaacgagacgcagagagggccctaccggccgcccagcctcaaccccctcgacccatccgcgccggactcgctgcggcgcatgtatgccaagttccagcaccagctcacgcgcacggcggagtcggcggccagcgggcagcggccctccaagcggacaacgatggaggcagcagcgcagcaggcacgggaggacatgcaggtgtgcggccagggccagggcggctcctctgacaacgaggaagtcgagagcaacatgggggtggttgacgcgctgctcggcggcatggcgcaactcgccggtgatgacacggcggaggtggtgcagcgccgccacgtgcaccgccgccggccgtcctcctgcgcaacaggaggcagtacgcaggacggcccgctcgcgcgccgacccgccgccggcagcggatgcggcagcgcgccagatgctccacagcaagatggggcatcgccagacgctccttggcaacacgaggcaccgccccgaggcaatggccaagcaaagcatgcggctctgcagccaagcaacgcatggggctcttga

SEQ ID NO: 5 - protein, 221 aa, Oocystis sp.
MAMPINPYNAKHETAVVWFRRDLRISDNPALAAALQSATNVIPLFIWAPEEEGQFQPGRANRWWLRNSLLSLSAELERLGSRLVCMAAPDCQSALCHVLASTGASALFTNRLYDPITMVRDNELLARVAGLGVRCFMFNSDRAVRAMGGAGPGGQAVQLVRRVLGQVPFALLTGTRAQGDAHAAPAPASDPGARGAPHRAWQRVRHAGRLPWPTDGGGAAD

SEQ ID NO: 6 - DNA, 2092 bp, Oocystis sp.
  source 1..2092: mol_type = other DNA
atggcgatgccgataaacccgtacaatgcaaagcacgagacagcggttgtatggttcaggcgggacttgcgcatctctgataacccggctctagccgcagcgctccagtcggcgaccaacgtgatcccgctgttcatatgggcgccagaggaggagggccagttccagccgggccgtgctaaccgatggtggctgcgcaactcgctgctgtcgctgtccgccgagctcgagcggctgggcagcaggctggtgtgcatggccgcgcccgactgccagtcggcgctctgccacgtgctggccagcaccggtgccagcgcgctgttcaccaaccgactgtacgatcccatcaccatggtgagggacaacgagctgctcgcacgcgtggccggcctgggtgtgcgctgcttcatgtttaactccgaccgtgctgtacgagccatgggaggtgctggaccaggcgggcaagccgttcaactcgttcgacgcgttttggggcaggtcccctttgcccttctcactggaacacgcgcacagggtgacgcacatgccgcacccgcccctgcctccgatcccggcgcccgtggcgctccccaccgtgcctggcagcgtgtgcggcacgctggtcgactcccttggcctactgacggaggaggagctgctgactaaccagcagctggagttcacgtggtccccgggcagtgccggtgcgcacaagctgtttgccaagtttgtgtccaggcgtcttcgccagttccagcacgatcgggccaagtcagaccggaacagcacctcgcgcctgtcgccgcacgtgcacttcggcgagatcagcgtgcggtacatgtactacgtggtcaagcagaaggaagccgagtggctggcgacgggagagcgcatcaccagctgtgcggacttcctgcagcagatggggtaccgcgagtactcgcgctacctctcctggcacttccccttcacccacgagcgctccctgcttgagcacgtgcgcgcctgcccgtaccgcatggaccaaaggctgttcaaggcttggcggcagggcaacaccgggtaccctctggttgacgcggccatgcgagagctgtggagctccggctggatgcacaaccgcatgagggtgaccgctgcttcctttttggtcaagcacctgctgctgccctggcagtggggcctgaagcacttttgggacgcgctggtggacgcagatctggaggccgatgcactggggtggcagtactgcgcaggctgcctggcagacgcacaccccctggactacatgatcgaccacgggaccgagtccaagcggtttgaccccgacggccagtacgtgcgccgctggctgcccgtgctggcaagggtgcccaggcaatacatacacgagccgtggaccgcaccgcagcatgtgctggacgaggcgggcgtcgagctcggctccaactacccctggccggtggtcgacgcgggggaggcgcaggtgctgctgggcgctgccaaggacgtggtggacgccgccctggaggcgctcaacgagacgcagagagggccctaccggccgcccagcctcaaccccctcgacccatccgcgccggactcgctgcggcgcatgtatgccaagttccagcaccagctcacgcgcacggcggagtcggcggccagcgggcagcggccctccaagcggacaacgatggaggcagcagcgcagcaggcacgggaggacatgcaggtgtgcggccagggccagggcggctcctctgacaacgaggaagtcgagagcaacatgggggtggttgacgcgctgctcggcggcatggcgcaactcgccggtgatgacacggcggaggtggtgcagcgccgccacgtgcaccgccgccggccgtcctcctgcgcaacaggaggcagtacgcaggacggcccgctcgcgcgccgacccgccgccggcagcggatgcggcagcgcgccagatgctccacagcaagatggggcatcgccagacgctccttggcaacacgaggcaccgccccgaggcaatggccaagcaaagcatgcggctctgcagccaagcaacgcatggggctcttga

SEQ ID NO: 7 - protein, 60 aa, Oocystis sp.
MAMPINPYNAKHETAVVWFRRDLRISDNPALAAALQSATNVIPLLHMGARGGGPVPAGPC

SEQ ID NO: 8 - DNA, 2092 bp, Oocystis sp.
  source 1..2092: mol_type = other DNA
atggcgatgccgataaacccgtacaatgcaaagcacgagacagcggttgtatggttcaggcgggacttgcgcatctctgataacccggctctagccgcagcgctccagtcggcgaccaacgtgatcccgctgttcatatgggcgccagaggaggagggccagttccagccgggccgtgctaaccgatggtggctgcgcaactcgctgctgtcgctgtccgccgagctcgagcggctgggcagcaggctggtgtgcatggccgcgcccgactgccagtcggcgctctgccacgtgctggccagcaccggtgccagcgcgctgttcaccaaccgactgtacgatcccatcaaccatggtgagggacaacgagctgctcgcacgcgtggccggcctgggtgtgcgctgcttcatgtttaactccgacctgctgtacgagccatgggaggtgctggaccaggcgggcaagccgttcaactcgttcgacgcgttttggggcaggtcccctttgcccttctcactggaacacgcgcacagggtgacgcacatgccgcacccgcccctgcctccgatcccggcgcccgtggcgctccccaccgtgcctggcagcgtgtgcggcacgctggtcgactcccttggcctactgacggaggaggagctgctgactaaccagcagctggagttcacgtggtccccgggcagtgccggtgcgcacaagctgtttgccaagtttgtgtccaggcgtcttcgccagttccagcacgatcgggccaagtcagaccggaacagcacctcgcgcctgtcgccgcacgtgcacttcggcgagatcagcgtgcggtacatgtactacgtggtcaagcagaaggaagccgagtggctggcgacgggagagcgcatcaccagctgtgcggacttcctgcagcagatggggtaccgcgagtactcgcgctacctctcctggcacttccccttcacccacgagcgctccctgcttgagcacgtgcgcgcctgcccgtaccgcatggaccaaaggctgttcaaggcttggcggcagggcaacaccgggtaccctctggttgacgcggccatgcgagagctgtggagctccggctggatgcacaaccgcatgagggtgaccgctgcttcctttttggtcaagcacctgctgctgccctggcagtggggcctgaagcacttttgggacgcgctggtggacgcagatctggaggccgatgcactggggtggcagtactgcgcaggctgcctggcagacgcacaccccctggactacatgatcgaccacgggaccgagtccaagcggtttgaccccgacggccagtacgtgcgccgctggctgcccgtgctggcaagggtgcccaggcaatacatacacgagccgtggaccgcaccgcagcatgtgctggacgaggcgggcgtcgagctcggctccaactacccctggccggtggtcgacgcgggggaggcgcaggtgctgctgggcgctgccaaggacgtggtggacgccgccctggaggcgctcaacgagacgcagagagggccctaccggccgcccagcctcaaccccctcgacccatccgcgccggactcgctgcggcgcatgtatgccaagttccagcaccagctcacgcgcacggcggagtcggcggccagcgggcagcggccctccaagcggacaacgatggaggcagcagcgcagcaggcacgggaggacatgcaggtgtgcggccagggccagggcggctcctctgacaacgaggaagtcgagagcaacatgggggtggttgacgcgctgctcggcggcatggcgcaactcgccggtgatgacacggcggaggtggtgcagcgccgccacgtgcaccgccgccggccgtcctcctgcgcaacaggaggcagtacgcaggacggcccgctcgcgcgccgacccgccgccggcagcggatgcggcagcgcgccagatgctccacagcaagatggggcatcgccagacgctccttggcaacacgaggcaccgccccgaggcaatggccaagcaaagcatgcggctctgcagccaagcaacgcatggggctcttga

SEQ ID NO: 9 - protein, 138 aa, Oocystis sp.
MAMPINPYNAKHETAVVWFRRDLRISDNPALAAALQSATNVIPLFIWAPEEEGQFQPGRANRWWLRNSLLSLSAELERLGSRLVCMAAPDCQSALCHVLASTGASALFTNRLYDPINHGEGQRAARTRGRPGCALLHV

SEQ ID NO: 10 - DNA, 2092 bp, Oocystis sp.; identical to SEQ ID NO: 8
  source 1..2092: mol_type = other DNA

SEQ ID NO: 11 - DNA, 23 bp, Synthetic construct
  source 1..23: mol_type = other DNA
tctggcgcccatatgaacagcgg

SEQ ID NO: 12 - DNA, 23 bp, Synthetic construct
  source 1..23: mol_type = other DNA
ccatggctcgtacagcaggtcgg

SEQ ID NO: 13 - DNA, 23 bp, Synthetic construct
  source 1..23: mol_type = other DNA
actgtacgatcccatcaccatgg

SEQ ID NO: 14 - DNA, 1643 bp, synthetic construct
  source 1..1643: mol_type = other DNA
atggccgagccggacgcgaacggcgcgtcggatggcaagcgcgcggagatttacacgtacgagttccccaacctcgtttactccatgaactggacggtaagcaagcacagttgtagcgcacacagccttgggtgctgctgcgtccttgctgatggtcatgcttgcgtcggccacccgttttctcacctaccgaaactggctgcgcacccctttcagttatgcttgtaatcaccgcctcattacctttgtgtgcagtctcgtcgggacaagaagtttcgactggcagtgggcagcttcatcgaggactataataacgtcgtcaatatcatatcctgtacgcgccttccccgcactacccagcgtagcggctcagtctgtcgtgcggggttggcacgacagtcgttgggttgaagactatttgaactattgctgattagtgacgcccatgctctctcgtaaagcggggcggatggctcctttcgcgcccactaccatctggccgcgacctgtggctctgacgcctcgctgggtctcgacctgctgaccatactccctcgttactggctgcaacgcagtggacgaggaacagggcaagtttgtatgcgacccgtcactgaccttcaagcatccgtacccaccgaccaaggtgatgtttgtgccagaccgggaaggcactcggcccgacctgttggccaccaccggcgactatctgcgcgtgtggaagatcggggaggatggcgtcacgctgcagaagctgctgaacgatgtaaggcctagattaacgtccagcgctgtggggaaggaccgacggcacgggccggaaagggacatgcatgccgtaccgggggtgatcacgggcgacggcccaccggatcatcatcttcctcgcttccaccaaccctgcgcaacgtccttcgcaacgtcttgaacaatattttgcatctttcacgttcatccatcctcgtcatggcgaaattaacttgcagaacaagaacagcgagttttgcgcgccgctcacatcgttcgactggaacgagaccgaccccaagcgcctgggcaccagctctatcgataccacgtgcacgatctgggacatcgagaagggcgtggtggacacgcagctcatcgcgcacgacaaggaggtgtatgacatcgcgtggggcggcgtcggcgtcttcgcgtcggtgtccgccgacggctcggtgcgagtcttcgacttgcgagacaaggagcatagcacgatcatctacgagacgccgtcgccagagacgccgctgctgcgcctggggtggaacaaacaggaccccaggtacatggcgacgatcgtgatggactctaaccgcgtggttgtgctggacatccgcgtgcccaccgtgcctgtcgccgagctgcagcggcaccaggcgtgcgcaaacgcgcttgcctgggcgccgcacagcagctgccacatctgcacagcgggcgacgacgcacaggcgctgatctgggacctcagcgcggtatccaaggagggcgactcgggcctggaccccatcctcgcgtacaatgcaggtcaagaggtaaaccagctgcagtggtcttcgacgcagcccgattgggtggcagtctgctttggcaacaaggcgcagatcctgcgagtgtga

SEQ ID NO: 15 - DNA, 2907 bp, Oocystis sp.
  source 1..2907: mol_type = other DNA
atggaggtcgctacaaacggcgaccacgcgcagcaccacctcggcatgccgcacggcgcgcggtctgaatacagcagtggcagcgatatgtcgcgcggcggcggtcatgcgggcagcagcggcggccaggctcagcagcagcaggctcagcaggccgccgcagctggcgaggccggcccaccgcgcaagctggtgatccttggcctgccatattttacaagcgacgacactctgcatggctacttttctcagcttggtcaggtagaggaggcgctggtcatgcgtgatcacgcgtcgggtcgctcccgtgggttcggatttgtgacgtttattaccgccgaagacgctgcgcgcgtggctgggcgggagtacagcgtcgatggtcggcgatgcgaggcaaagttcgcgctgccgcgtggcgagagcgcaagccagcgtgttacgcgcatcttcgtggcaaagttgccaccgcacgttgccgaggacgagctgcgcacctactttgagcaggtgcgacgccccctggggctgggttccaacaactgccctagcaccggtcacaaggtgcctttaggcttctgcaggaatgtgcctccggttaatgtattgattcatcaaccttatcagcgtattggtgctgcattcaagtggtgatcaccacgtgcatctgccagctgtctctcgatcacgtctccaaacggcgatccgcctagtccgcccaagccctgcccaagcgcgtcgtcgtgctgggccccccgttccgtgtgcaagacggtgtgcttgcttacgaggtgtataccgtttttattttcttacgcgcagtacggagcgattcaggatgtgtacatgcccaaggatgcttccaagcaggcgcgacgtgggattgggttcgtgacgttcgcgagccccgaggcggttgacgccgtcattcgcacatcccacgtgttacacggccaggagctcgtcgtcgataaggccgcgccaaagcagaaagagccgtttccgcttggagctggcctaccaggcgccacagccagtggcgtgccttaccggtcggcgcagccgtcgctgtcagccgaacggcttgccagcttgagcaacggcgcattcgccggcttgattcccggcggctacggcttcggcggtcatgggctgcagcagcagcagcacatactgccggggtcaatggctggtggcgcgctcagcggttctgcaggatcgctatacgactcgttcggaggcgcgatcaacggtactcaagaagacggaaacggagacatgggcctttccggtaagcacttgctttttgcgtgcaatctagataaagggatatttacgttcatccatacttgtattcgcacccaggaaaatccaggcagttgcctgatctcacgtatttttggtgcacgccacgccccaagtggaaaggactatggaattattaaagagtctgcgcagaacagacgctagttccttcggttctgtcagaccctacacttgctttgctgcacgccacgccccaatcaacgtatccccctcggtgtgcactgcgctgcccgtttccttgcgcaggtctgggatttgtggaccacaacgcaaacggtggcctgcacggtctgcacggcttgcaaggatccaacccaggcatgcagtatgctattgcacagctggcccgtgcgcagcaggcggcgcagcttggtacgtgccgccgctgctgaacctcaacccgcctcccacttcatcacgctccgttcttattgcttctttgtgcctagtatatcccatgcctcgatggcgtctcgcagtcaataacccacacaccaacacactaacgaattctgcatcacttgtgcggcaggtctctcgctgacatttgacggtcgcccaagcagctccctcaacctgggcgcagctgcgacggccgcggccgcacagaatggcggccggccggcgggcgcgccgggcagcgccaactcgctcacagacctcgaccggatgtatggcgtgcagcagcagcagcaagcaggtgcgtgtggcgttctgcccagcttggctggaattattgcgcgaaaggacgttctattgatgttctagacaaatgacctgggttggaactatctcgttttttttttaacgtgaccaagaggcctgtcgtgccaggaattgctagcgagcatggtttgagcacgagcgcgtacgctcaccgcagttcctcgctcagtccagccgcctgggtttgtacgtggcctctgcatcgtttgagcgctgcatcctcctgctccgttgctcaccccacctcgcccccacccaccccctgcagggctttccactggcctgcccaacggcctggtctcgctgcgcggcacaggcgcaggcggcccgaccagccccggtggtggacgcggccctggcggcattggcgctgagccggtcgccgcaggtggtaccagtgctggcgccggcggggcactgtgcaccaaccgcgtgttcattggcaagctgggcaaggatgtgatggaggcggacattaaggagtactgctcgcgatttgggtacgtgctggacgtgtacatcccgcgcgacaaaaacaacaagcgagagcatcgcggctttggctttgtgaccttcgagaccgaggccgcggtcgatcgcatccttgcgtttgatgaccaccaaatccacggctcggtgattgccgtcgaccgagcgctgccgaggcaggaggacacgagccagagcagcgtggcgctcagtggtgaccagcagtatggcgctgacgtcagcagtgacgctgtcagcgccgcactcgggatggccgcgcttggcctgggcgccaacggacaggtgctgccggggcctgcgcgccacaacaacgaccgcaaccggtatctgtaccagccctactag

SEQ ID NO: 16 - DNA, 1566 bp, Oocystis sp.
  source 1..1566: mol_type = other DNA
atgcagtcgctgcacagccaggcgcccaccgtgggcaactgcgggcaactgtcgaggcaacaacggcgaaatgctggcctgggatcggccttctctggcggcccccttgctggcaaggcgtttctagcgggcagcaagctgaccgttcgggcaagcagcaagtctggcgtgaggggcgcgggcaagctgcaggtgcaagcagttatcaccaaacagagtgctgatcagggttatatgaccgagagcatctcgaccaggagtgtggcagcgatcatcctcggcggtggcgcgggcactcgcctgtaccccctgaccaagcagcgcgcgaagccggccgtgcctattggcggagtgtaccgccttatcgacgtgcccatgtccaactgcctcaactcggggatcagcaagatctacatcctgacgcagttcaactccacgtcgctgaaccgccacctgggccgcacctacaacttcggcaacggccctcgcagcggtggcgatggcttcgttgaggtgttggcagccacgcagactcccaccgacgcaacctggtttcagggtaccgccgatgctgtgcgacagtacacgtggctgctgcaggatatcaagaacaggacagtggaggacattgtgattctgtcgggagaccacctgtaccgcatggactacatgaaattcgtgaaccagcaccgtgagaccgggtcagacatcaccgtcggctgcctgccttgcgatcccgagcgtgccgcggactttggactcatgaagatcgacaatgagggacgcatcgttgagtttgctgagaagcccaagggtgacgcgctgcaggcgatgaaggtggacaacacactgctggggctgtcaaagacggaggctgcggagaagccgttccttgcgtctatgggcatctacgtgttcaagaagtctgcgcttattgacctgctgaccaaggagtaccccaacgacaacgactttggcggcgagatcatccccaaggctgcggcaggagactacaaggtgtctgcctacctgttcaacggctactgggaggacatcgggaccatcaagtcgttcttcgacgcaaacctggcgctggcgcagcacccgccgcgctttgagttctacgacccgcagcagcccatctacaccagcccgcgcttcctgccgcccgcaaagatagtgcggtgcaaggtgcaggacgcgatggtctcgcccgggtgctacctggcggactgcacggtggagaatgccatcatcgggctgcgcagccgcatcgagaagggcgccgtcattcgggacgccatggtgatgggcgcggactactacgagaacgatgcgcagcgcgccgcactcaacgcggcgggccgcgtgcccatcggcgtgggtgaaaacagcaccataatgaacgccatcctggacaagaacgcgcgcattggcaagaactgctccatcgtgaacaaggacggcgtcgacgaggccagccgggaggaggacggcttcttcatccgcagtggcatcatcactgtctgccgcaacgccgagattctggacggcacagttgtctaa

SEQ ID NO: 17 - DNA, 3751 bp, Oocystis sp.
  source 1..3751: mol_type = other DNA
atgcgcgatcccagtgggagtgccacgcgacctactcctgctcgggttcacctggcggggaccccagcccatgcaaaccaatgccccaggatttcagcgtgtctgatcagcaggtaacggcgcctttgacgcgcctgatgccgctggagctgcgtgtacgacattgccgcacgatctagtggcgctcacgactgcagggacatggtagcccaggaacacggagacgggccgcccaggtggcacagccatcccagacatcggctgtgtccagtagttcgtgtgtatcttcgccacgtatcaacggtgcggtggaggcgttggaccgacacaagccagcgagcaacgcacgcgttgcatcagctgcagcaggccaatcaagcaccggagcagcggcacaggcacagcgcaatttgctcacgaagcctttcaagggtctgcataacgacggtcatacaacgagaactgggacttgatcatatgtgtcggggacgagtttgtgtccagctccaacctcaagtacgtcgtcatcgacttgctgggccagggcacgttcggccaggtagtgcgctgctggtgcgaccagacgcaggagtatgtagccgtcaaggtgataaaaaatcagccggcttactatcagcaagcgcgcgtagaggttggcctgttgcaatacctcaaccgctgtgcggacgcggacgacgtccggcacattgtgcgcctccgcgactactttttgttccgtaaccacttgtgcctcgcgttcgagctgctgtcggtcaacctgtacgagctcatcaagcacaaccagttccgtgggctgtctgcggggctcgtgcgcgtgttcatcgctcagctgcttgatgcgctggtggtgctgcgtgagtctcgcctcatccactgcgacctcaagccggagaacgtgctgcttacgggcgctgagtcagctgacataaaggtcatcgactttgggtccgcttgcctggagagcaaaacggtgtacagctacatccagagccgcttctatcgctctccagaggtggtgcttggctacccgtacaacgtggcgatcgacatgtggtccctgggctgcatggccgcggagctgttccttggcctgccgctgttcccgggcgcatcggagcacgatttgttgtctcgggtggtgcaggcggtgggcctcccgccgctgtacctgctgcagggcgcaaagcacaccaacaagtatttcaagatggtggagcgcgtggtgcggctgccaagcggcaggtctgaggtggtgcccgagtatgtgatgcgcactgccgcggagtttgaggctctcacggggctcaaggccaccaccggcaagcgctacttctcacatacgcgcctgcaggacatcatcaactcatacccttcagagggcgcgggcagcgagctgcgccgctcccttctggacttccttcggggcgtgctggaccccgacccagcggcgcgctggacgccgcagcaggcggcgcgccacccgtttgtgacgggccagccgttcgcggcgccgttccagccagacggggagcgtacgcccccgcctcccggctgggacgcctccgcggccggcatgctgtccagcagtgctgggcaccaggccgctcagcagcagcaggcagggtggaacggcgcgtccgcgtcgccgcactatggcagcgcggcggcggcgatgctcgccacgtcgccgcacgtgcaggcgcatgtggcggccatggcggcgctcgcccagcagcagcagcagcagggcatgggcaccccgtcagggctgccctcgtaccgcccgcagcctgtgcctgtaccgcgtgcggggcccggctacggcggtggcggcggctgggcagcgccgtatccccaccagcaccagcagcagcagcaggccggcagccttacgcagcagtacctgcagcagcagcagcagcaggcaggcggcagcctgggcctgttcagcccgcccggcctgggccccgccagcctgatgcagctcgaccacctgagcgcggcctcgggctcgttcttcagcccgcctgggtcgctggtcaacgcgaccgggcggctggcgccagcgccgggggccgcgcgctacggcagctaccagccgatgccggtggacatgggcttcagccccgccggcagcctgtcggcaaactcggtgctggcggtggccatcgcggcagccaacgccgcagccgcctcggccgctgcgcagcagcagcagcagcagcacggcgtcgcggagcagctgcggttgcagtggcagggcgccgccgtgggcggcggcggcggcggcggcttccccagtgggctggcgcgcatgtcgggcgtggcagggggctcgtactccggccagtctgcagtgccgatggtggggtcgtatgccagcagcctgccggggggcagcgccgccaccgcggcggcgggcccaggcacgggctcaaacccgctggccatgctcgcggcgcagcaggcggccgaggacgcactcgccagcagcctgcggcgcgggtcgatgggcgcaggcgcgagcggcgggtatggcgccggcttcggcggctccgcagcggccacggccgctcccgccgccgccgcggcggcggcccagcagcctgcgccggtggacatgtcggctgctgcaggcagcctcggcgcggtgctgcagcagctgcaggcgacacaggccgcggcgcaggcgcaacagtttgtggcggcggccgccggtggcgggagccgcggccgtacgccgccgccgccggcggacggtgcgcttgccaccgcttgcgctgcagctgctgcgggagcagtggccggggggggcggcagcgcccaccagcagcagcagcggcacgagtcggtgtcgccatctcctggggactgggacccgctgtacagcgacgaccagctcctggaggacgatacgcccgctcagcagcgtacaccgccgcaacaaccgcagcagcgcaagggcagcggcaacttggcagcggcgctggcagcggccgtcacgctgcctgcccagcagcagcagtggcagcagcagcagcagcagcggcagcccctcgcgggcgggctgtctccgccagaccctgcgctaacagcggcagccgcggcggcacttggcgcgctgcagcagcagcagcagcagcagcaggccacggcaggtgccggtgcggccacactggacaccgcagacctagtgggctggctgcagcagcagcagctgctgcgcgcgctgcgcccgcctccggcaacggcagacgcccgcggcgccggaggtgccgccgctgccgcggccgcctccattgagcctgctggtgtggcgggtgccgtgtcggcggcggcgccggtgcccggcgaccaacagcaccgtgcagcagggccaatgccgcccctctctgctgccgagcagcagcaaacacaggccggtagcagcgggggcgctgctgcccatgctgcggcagcagcaggggaaggtgttgccgccggcctgggcggcggtgctggtggcgcctctcggcggaacagttttgagattgatcggaggggtagttttgagattggctttgaggttgctccagtcgtgtccacaggcctgcagcaggcgtcgggagcgcgcactggcgcggcacccgcccacgccgggggtggattcagctgcttgtctgcgcagctccacggcagcaggcagcacaagcagcagcagccggaagcggcagaacattccagcaagtag

(19) United States
(12) Patent Application Publication
(10) Pub. No.: US 2025/0092430 A1
(43) Pub. Date: Mar. 20, 2025

(54) Recombinant Algae Having High Biomass and Lipid Productivity

(71) Applicant: VIRIDOS, INC., La Jolla, CA (US)

(72) Inventors: Eric Moellering, La Jolla, CA (US); Mark Moosburner, La Jolla, CA (US)

(21) Appl. No.: 18/887,921

(22) Filed: Sep. 17, 2024

Related U.S. Application Data
(60) Provisional application No. 63/539,076, filed on Sep. 18, 2023.

(51) Int. Cl. C12P 7/64; C12N 9/88

(52) U.S. Cl. CPC C12P 7/64; C12N 9/88; C12Y 401/99003

(57) ABSTRACT

The invention provides a recombinant photosynthetic organism that has been genetically modified in a gene encoding a protein kinase-like protein. The recombinant organism exhibits higher biomass productivity and higher lipid productivity versus a corresponding control algal organism not having the genetic modification. The recombinant organism is therefore useful in applications requiring biomass and/or lipid productivity, e.g., in the production of biofuels or other lipidic matter. Methods of using the organism and biomass containing or produced by the organism are also provided.

### CROSS-REFERENCE TO RELATED APPLICATIONS

This application claims benefit of priority under 35 U.S.C. § 119 (e) of U.S. Provisional Application No. 63/539,076, filed Sep. 18, 2023. The disclosure of the prior application is considered part of an is herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name SGI2330-1, was created on Sep. 2, 2024, and is 37 kb.

### FIELD OF THE INVENTION

The invention involves recombinant algae and methods for producing biomass and lipids.

### BACKGROUND OF THE INVENTION

The production of biofuels presents great opportunities to develop environmentally sound sources of energy that can be obtained at reasonable cost. Efforts have been directed towards using algae or other microorganisms to produce hydrocarbons that can be used as biodiesel or other biofuels due to their high lipid content. Additional specialty chemicals can also be obtained from these organisms and for use in consumer products.

Since algae use energy from sunlight to combine water and carbon dioxide to produce biomass, achieving increased productivity offers the possibility of a carbon neutral fuel source. The development of algal strains with very high lipid productivity for the production of algal-sourced biofuels therefore presents the possibility of a significant reduction in new carbon dioxide released into the atmosphere and a consequent reduction in the problem of global warming.

The development of commercially viable algal biofuels requires strains with high lipid and biomass productivity. Even the most productive wild type strains are not sufficiently productive to permit an economically viable development of this resource. Strategies for increasing algal production of biofuels and other products have included modification of nutrition provided to the organisms, such as cultivating the organisms in nitrogen, phosphorus, or silicon deficient media. Other strategies have included modification of cultivation conditions or environmental protocols, or various efforts directed towards genetic engineering of the organisms. While engineering algae strains to have a combination of increased photosynthetic efficiency (resulting in increased overall biomass productivity) and/or high lipid productivity could provide a solution to this problem, deficiencies still remain. The development of higher performing strains continues to be a barrier to efficient utilization of this energy source.

### SUMMARY OF THE INVENTION

The invention provides a recombinant photosynthetic organism that has been genetically modified in a gene encoding a cryptochrome/photolyase FAD-binding domain. The recombinant organism exhibits significantly higher biomass productivity and higher lipid productivity versus a corresponding control algal organism not having the genetic modification. The recombinant organism is therefore useful in applications requiring biomass and/or lipid productivity, e.g., in the production of biofuels or other lipid-containing matter. Methods of producing and using the organism, and biomass containing or produced by the organism is also provided.

In a first aspect the invention provides a recombinant photosynthetic organism having a deletion, disruption, or inactivation of a) a gene encoding a cryptochrome/photolyase FAD-binding domain and having a nucleic acid sequence having at least 80% sequence identity to the nucleotide sequence of SEQ ID NO: 2; or b) a gene encoding a cryptochrome/photolyase FAD-binding domain having at least 80% sequence identity to the polypeptide sequence of SEQ ID NO: 1. The recombinant photosynthetic organism exhibits higher biomass productivity and higher lipid productivity versus a corresponding control algal organism not having the deletion, disruption, or inactivation.

In one embodiment the gene encoding the cryptochrome/photolyase FAD-binding domain encodes a polypeptide sequence having at least 90% sequence identity to the polypeptide of SEQ ID NO: 1 and/or a polypeptide sequence having at least 95% sequence identity to the polypeptide of SEQ ID NO: 1. In any embodiment the recombinant photosynthetic organism can be a Chlorophyte alga. In any embodiment the organism can be of the Class Trebouxiophyceae. In one embodiment the deletion, disruption, or inactivation is to a regulatory sequence of the gene encoding the cryptochrome/photolyase FAD-binding domain. The regulatory sequence can be a promoter. In one embodiment the deletion, disruption, or inactivation can be a deletion of one or more amino acids of the encoded cryptochrome/photolyase FAD-binding domain. In one embodiment the deletion, disruption, or inactivation is a disruption by an insertion in the gene encoding the cryptochrome/photolyase FAD-binding domain. The insertion can involve insertion of a stop codon in a sequence encoding the cryptochrome/photolyase FAD-binding domain. In any embodiment the recombinant photosynthetic organism can have at least 20% or at least 25% higher lipid productivity versus a control photosynthetic organism.

In any embodiment the recombinant photosynthetic organism can have at least 35% higher biomass productivity per unit time versus the corresponding control organism. In any embodiment the recombinant photosynthetic organism can have a FAME/TOC ratio of at least 0.4 after two days of cultivation. In any embodiment the recombinant photosynthetic organism can have a higher biomass productivity under nitrogen deficient conditions. In any embodiment the recombinant photosynthetic organism can have a higher total organic carbon production under nitrogen deficient conditions.

In various embodiments the recombinant photosynthetic organism is from a family selected from Oocystaceae, Chlorellaceae, or Eustigmatophyceae. The recombinant photosynthetic organism can be an alga of the genus selected from Chlorella, Parachlorella, Picochlorum, Tetraselmis, or Oocystis. In one embodiment the recombinant photosynthetic organism is an alga is from the genus Oocystis. In various embodiments the gene encoding the cryptochrome/photolyase FAD-binding domain has a nucleic acid sequence having at least 90% sequence identity to any one of SEQ ID NO: 2, 4, 6, 8, or 10. In one embodiment the recombinant photosynthetic organism does not contain any other engineered deletions, disruptions, or inactivations besides the deletion, disruption, or inactivation of the gene encoding a cryptochrome/photolyase FAD-binding domain.

In another aspect the invention provides a biomass product containing a recombinant photosynthetic organism described herein.

In another aspect the invention provides a recombinant photosynthetic organism having a genetic modification to a gene encoding a cryptochrome/photolyase FAD-binding domain that has a polypeptide sequence having at least 90% sequence identity to SEQ ID NO: 1, or a gene encoding a cryptochrome/photolyase FAD-binding domain having a nucleic acid sequence having at least 90% sequence identity to SEQ ID NO: 2. The recombinant photosynthetic organism exhibits higher biomass productivity and higher lipid productivity versus a corresponding control photosynthetic organism not having the genetic modification.

The recombinant photosynthetic organism of any one of claims 23-24 wherein the recombinant alga has at least 35% higher biomass productivity per unit time versus the corresponding control photosynthetic cell or organism.

In another aspect the invention provides methods of producing a composition containing lipids involving steps of performing a genetic modification in a photosynthetic organism to a gene encoding a cryptochrome/photolyase FAD-binding domain having a polypeptide sequence having at least 80% sequence identity to SEQ ID NO: 1, or a gene encoding a cryptochrome/photolyase FAD-binding domain having a nucleic acid sequence having at least 80% sequence identity to SEQ ID NO: 2. The recombinant photosynthetic organism exhibits higher biomass productivity and higher lipid productivity versus a corresponding control photosynthetic organism not having the genetic modification. The method can also involve a step of cultivating the organism and thereby producing a composition containing lipids. In one embodiment the methods also involve a step of harvesting a lipid containing composition from the photosynthetic organism. In one embodiment the lipid containing composition harvested is at least 50% lipid (w/w). The genetic modifications to the sequence encoding the cryptochrome/photolyase FAD-binding domain can be any described herein. In one embodiment the recombinant alga has at least 50% greater lipid productivity versus a control alga.

In another aspect the invention provides a method of producing a recombinant photosynthetic organism. The method involves performing a deletion, disruption, or inactivation of a gene encoding a cryptochrome/photolyase FAD-binding domain and having at least 80% sequence identity to the nucleic acid sequence of SEQ ID NO: 2, or a gene encoding a cryptochrome/photolyase FAD-binding domain having at least 80% sequence identity to the polypeptide sequence of SEQ ID NO: 1. The recombinant photosynthetic organism produced can exhibit higher biomass productivity and higher lipid productivity versus a corresponding control algal organism not having the deletion, disruption, or inactivation.

### DETAILED DESCRIPTION OF THE DRAWINGS

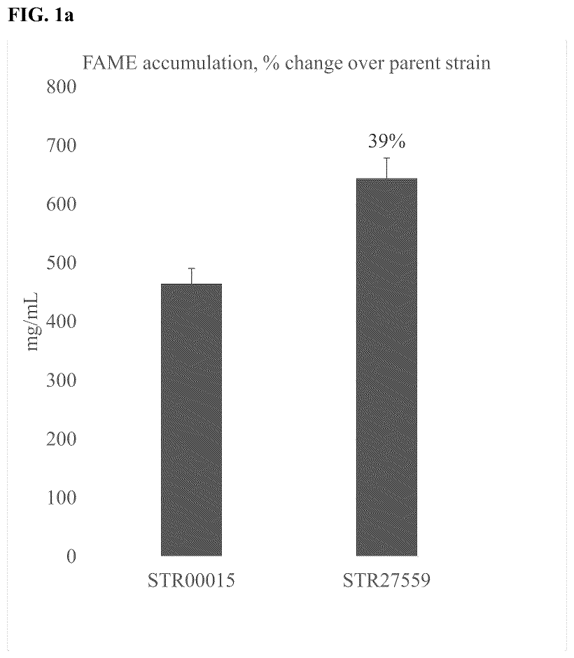

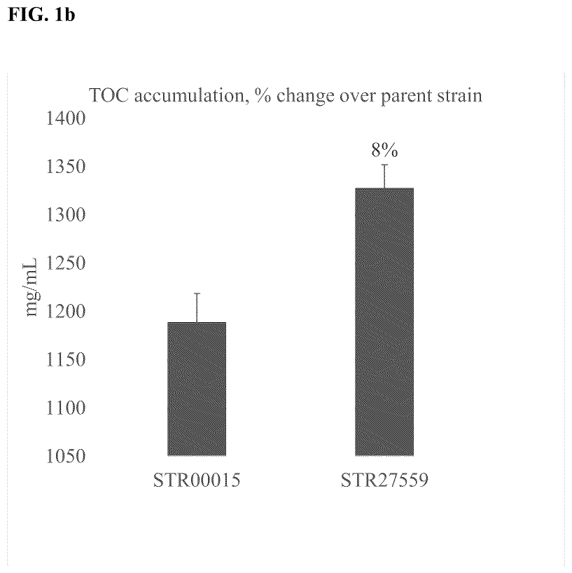

FIG. 1a provides a bar graph demonstrating an increase in FAME accumulation at 48 hours for the mutated strain (STR27559) versus the parent wild-type strain (STR00015). FIG. 1b provides a bar graph demonstrating an increase in TOC accumulation at 48 hours for the mutated strain versus the parent wild-type strain. FAME and TOC accumulation metrics are presented in absolute FAME and TOC (histogram bars) and the percentage change over the parent strain STR00015 (labels above mutated strain (STR27559) bars). FIG. 1c provides a bar graph demonstrating that the mutated strain has a higher FAME/TOC ratio than the parental wild-type strain. FAME/TOC metrics are reported as the fraction of TOC that is FAME at 0 hours, 24 hours, and 48 hours after nitrogen starvation and the percentage change compared to parent is labeled above the STR27559 bars.

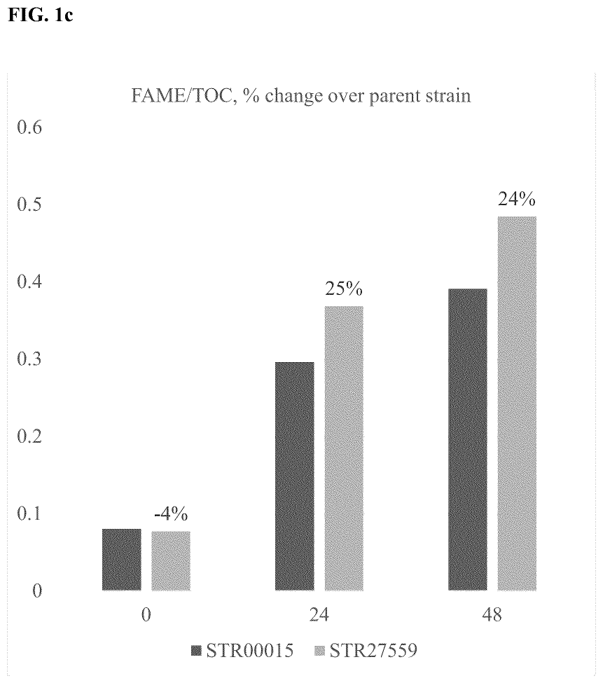

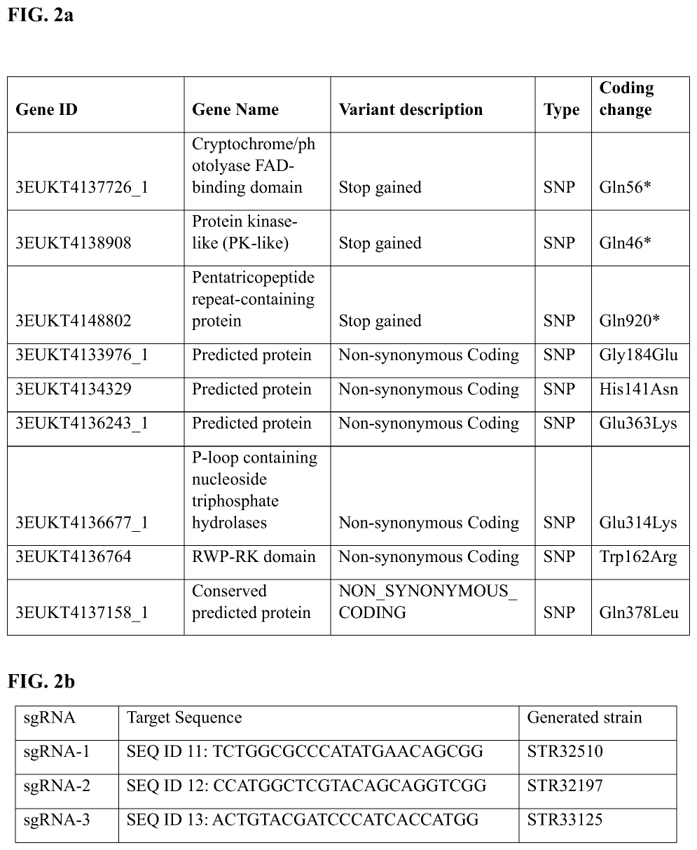

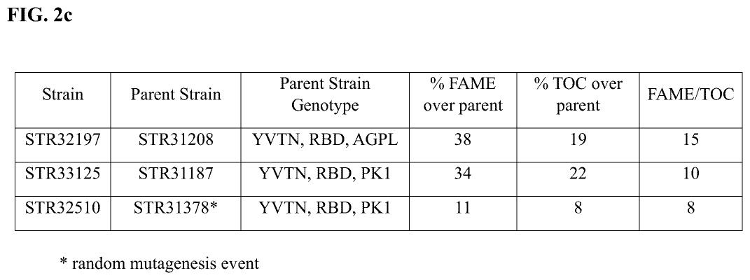

FIG. 2a provides a table of the list of small variants found in the mutated strain (STR27559) from ReSequencing analysis. Here, a subset of nine small variants are described including the one found in the CRY1 gene (first row). FIG. 2b provides a table of sgRNA target sequences used to generate the CRY1 knockout strain. FIG. 2c provides a table showing the percent improvement in FAME, TOC and FAME/TOC for each CRY1 knockout strain relative to its parent strain. The improvement metric was analyzed on day 5 of nitrogen starvation of a seven-day nitrogen starvation growth experiment. The genotypes for the parent strains are included as a list of the gene common names that are knocked out by Cas9-based methods. STR31378 has an asterisk (*) to indicate that the strain was mutated by random mutagenesis methods using uv light.

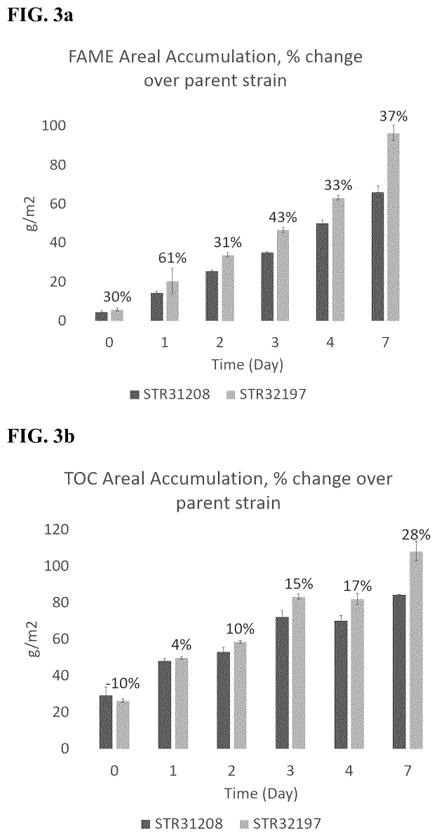

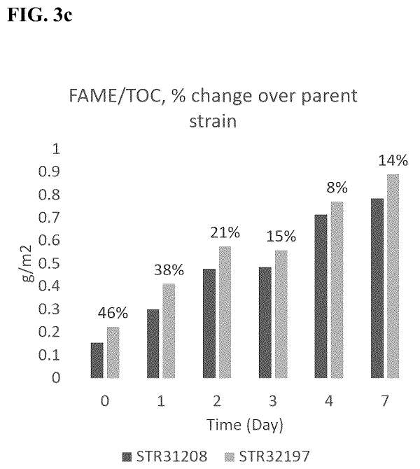

FIG. 3a provides a bar graph demonstrating FAME areal accumulation data collected from a seven-day nitrogen starvation growth assay of the Cry1 knockout strain (STR32197) compared to its parent strain (STR31208), which itself had been Cas9 modified to disrupt genes YVTN (SEQ ID NO: 14, WD40 repeat containing proteins), RBD (SEQ ID NO: 15, RNA-binding domain), and AGPL (SEQ ID NO: 16, ADP-glucose pyrophosphorylase)). FIG. 3b is a bar graph demonstrating TOC areal accumulation of the same. FIG. 3c is a bar graph demonstrating FAME/TOC carbon partitioning data of the same. In FIGS. 3a-b FAME and TOC areal accumulation metrics are presented in absolute FAME and TOC per square meter (histogram bars) with error bars representing the standard deviation from three replicate cultures. In FIG. 3c the FAME/TOC metrics are reported as the fraction of TOC that is FAME for all time points. For all charts, the percentage change observed for the Cry1 knockout (STR32197) compared to its parent strain (STR31208) is labeled above the knockout strain bars.

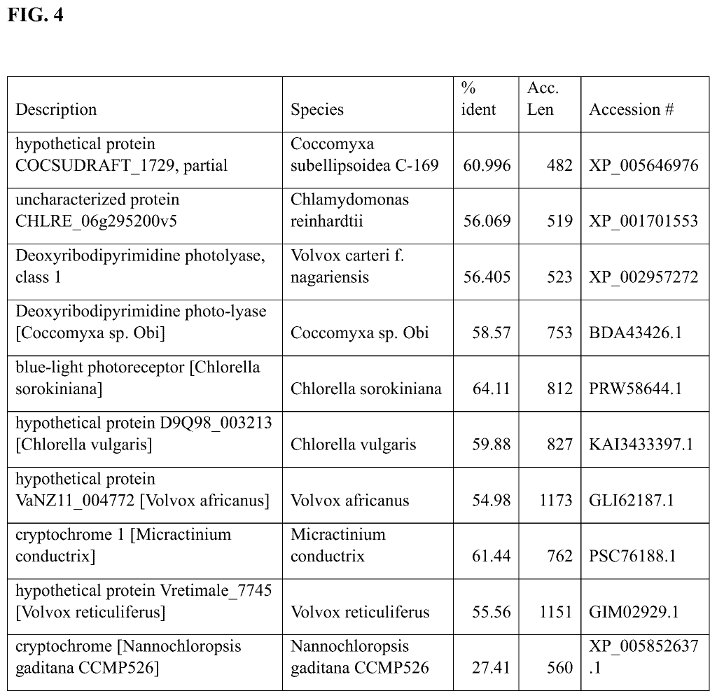

FIG. 4 presents a table showing the percent improvement in FAME, TOC and FAME/TOC for each CRY1 knockout strain relative to its parent strain. The improvement metric was analyzed on Day 5 of nitrogen starvation of a seven-day nitrogen starvation growth experiment. The genotypes for the parent strains are included as a list of the gene common names that are knocked out by Cas9-based methods. One strain (STR31378) has an asterisk (*) to indicate that the strain was created by random mutagenesis methods.

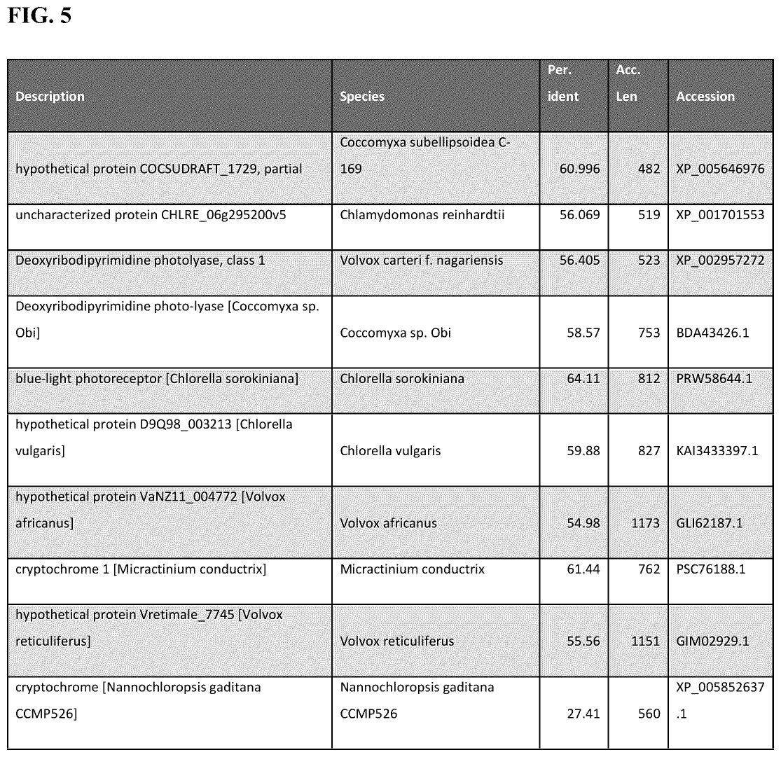

FIG. 5 presents a table showing Cry1 orthologs in green algae.

### DETAILED DESCRIPTION OF THE INVENTION

The invention provides a recombinant photosynthetic organism that has been genetically modified in a gene encoding a cryptochrome/photolyase FAD-binding domain. The recombinant organism exhibits higher biomass productivity and higher lipid productivity versus a corresponding control algal organism not having the genetic modification. The recombinant organism is therefore useful in applications requiring biomass and/or lipid productivity. In one embodiment the photosynthetic organism is an alga, such as a green alga. The invention shows that this single gene modification results in a strain having significantly higher lipid and biomass productivity. In any embodiment the genetic modification can be a deletion, disruption, or inactivation of a gene encoding a cryptochrome/photolyase FAD-binding domain.

The recombinant cell or organism of the invention having a genetic modification described herein can have higher lipid productivity (e.g., as measured by FAME) and/or higher biomass productivity versus a corresponding (control) cell or organism. In some embodiments the genetic modification is an attenuation(s) of a gene encoding a cryptochrome/photolyase FAD-binding domain. In any embodiment biomass productivity can be measured as the rate of biomass accumulation, for example as measured by the total organic carbon (TOC) content of the respective cells or organisms.

In one embodiment the lipid and/or biomass productivity is higher in batch culture, i.e. a culture where nutrients are not renewed or re-supplied to the medium during culturing, compared to a corresponding (control) cell or organism. Any of the mutant cells or organisms disclosed herein can be photosynthetic cells or organisms. Any of the recombinant (mutant) cells or organisms described herein can exhibit increased lipid productivity and/or increased biomass productivity under photoautotrophic conditions compared to a corresponding control cell or organism, i.e. conditions where the recombinant cells or organisms can produce their own biomass using light, carbon dioxide, water, and nutrients via photosynthesis. Corresponding (control) cells or organisms are cells or organisms that are useful for evaluating the effect of any one or more of the genetic modifications. Corresponding (control) cells or organisms are cells or organisms that do not have the one or more genetic modifications being evaluated and that are subjected to the same or substantially the same conditions as the test cells or organisms such that a difference in the performance or characteristics of the cells or organisms is based only on the genetic modification(s) being evaluated. In any embodiment the corresponding (control) cells or organisms can be of the same species as the test organism. They can differ only in the genetic modification(s) being evaluated. In some embodiments the corresponding (control) cell or organism is a wild-type cell or organism. But the corresponding (control) cell or organism can also be a laboratory strain or parental strain of the test cell or organism. Substantially the same conditions can be the same conditions or slightly different conditions where the difference does not materially affect the function, activity, or expression of the nucleic acid sequence modified.

In any embodiment the recombinant cells or organisms can be algal cells, e.g., a green alga. In one embodiment the recombinant alga has a genetic modification to a gene encoding a cryptochrome/photolyase FAD-binding domain. The lipid products of these mutants can be further processed into biofuels or used in the production of other specialty chemical products.

The genes encoding the cryptochrome/photolyase FAD-binding domain can be any of the nucleic acid sequences described herein, which can encode a cryptochrome/photolyase FAD-binding domain.

In various embodiments the encoded cryptochrome/photolyase FAD-binding domain can have a polypeptide sequence of any one of SEQ ID NOs: 1, 5, 7, or 9, or a polypeptide sequence having at least 80%, or at least 85%, or at least 90%, or at least 95%, or at least 98% sequence identity to any one of SEQ ID NOs: 1, 5, 7, or 9, which in any embodiment can be a sequence of at least 100, or at least 200, or at least 300, or at least 500, or at least 600, or at least 700, or at least 800, or at least 1000 amino acids; and/or the coding sequence of the gene encoding the cryptochrome/photolyase FAD-binding domain can have a nucleic acid sequence selected from SEQ ID NOs: 2, 6, 8, or 10, or a coding sequence (CDS) having at least 80%, or at least 85%, or at least 90%, or at least 95%, or at least 98% sequence identity to SEQ ID NO: 2, 6, 8, or 10, which in any embodiment can be a sequence of at least 100, or at least 200, or at least 300, or at least 500, or at least 600, or at least 700, or at least 1000 nucleotides. In some embodiments the sequence encoding the cryptochrome/photolyase FAD-binding domain can have any of the nucleic acid sequences described herein, and can encode any of the polypeptide sequences disclosed herein; the nucleic acid and polypeptide sequences are hereby disclosed in all possible combinations and sub-combinations.

In some embodiments recombinant cells or organisms of the invention can have a reduced amount of chlorophyll b, and can have an increased chlorophyll a to chlorophyll b ratio (chl a/chl b) compared to a corresponding control cell or organism. The recombinant cells or organisms can have decreased photosynthetic antenna size, for example reduced photosystem II (PSII) and/or reduced photosystem I (PSI) antenna size. In various embodiments the cross-sectional unit size of the PSII and/or PSI antenna of the recombinant cells or organisms disclosed herein can be reduced by at least 10%, at least 20%, at least 30%, at least 25%, at least 30%, at least 35%, at least 40%, at least 45%, at least 50%, or at least 60% compared to the PSII and/or PSI antenna size of a corresponding control cell or organism.

As used herein, “exogenous” with respect to a nucleic acid indicates that the nucleic acid has been introduced (e.g., “transformed”) into an organism or cell by human intervention. For example, such an exogenous nucleic acid can be introduced into a cell or organism via a recombinant nucleic acid construct. An exogenous nucleic acid can be a sequence from one species introduced into another species, i.e., a “heterologous” nucleic acid. A heterologous nucleic acid can also be an exogenous synthetic sequence not found in the species into which it is introduced. An exogenous nucleic acid can also be a sequence that is homologous to an organism (i.e., the nucleic acid sequence occurs naturally in that species or encodes a polypeptide that occurs naturally in the host species) that has been isolated and subsequently reintroduced into cells of that organism. In some embodiments an exogenous nucleic acid that includes a homologous sequence can be distinguished from the naturally-occurring sequence by the presence of non-natural sequences linked to the exogenous nucleic acid, which can include but are not limited to non-native regulatory sequences attached to the homologous gene sequence in a recombinant nucleic acid construct. Alternatively or in addition, a stably transformed exogenous nucleic acid can be detected and/or distinguished from a native gene by its juxtaposition to sequences in the genome where it has integrated. Further, a nucleic acid is considered exogenous if it has been introduced into a progenitor of the cell, organism, or strain under consideration.

A “recombinant” or “engineered” nucleic acid molecule is a nucleic acid molecule that has been altered through human manipulation. As non-limiting examples, a recombinant nucleic acid molecule includes any nucleic acid molecule that: 1) has been partially or fully synthesized or modified in vitro, for example, using chemical or enzymatic techniques (e.g., by use of chemical nucleic acid synthesis, or by use of enzymes for the replication, polymerization, digestion (exonucleolytic or endonucleolytic), ligation, reverse transcription, transcription, base modification (including, e.g., methylation), integration or recombination (including homologous and site-specific recombination) of nucleic acid molecules); 2) includes conjoined nucleotide sequences that are not conjoined in Nature; 3) has been engineered using molecular biology techniques such that it lacks one or more nucleotides with respect to the naturally occurring nucleic acid molecule sequence; and/or 4) has been manipulated using molecular biology techniques such that it has one or more sequence changes or rearrangements with respect to the naturally occurring nucleic acid sequence, or has a sequence (e.g., by insertion) not found in the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector.

When applied to organisms, the terms “transgenic” “transformed” or “recombinant” or “engineered” or “genetically engineered” refer to organisms that have been manipulated by introduction of an exogenous or recombinant nucleic acid sequence into the organism, or by genetic modification of native sequences (which are therefore then recombinant). Recombinant or genetically engineered organisms can also be organisms into which constructs for gene “knock down,” insertion, deletion, disruption, attenuation, or inactivation have been introduced to perform the indicated manipulation. Such constructs include, but are not limited to, RNAi, microRNA, shRNA, antisense, and ribozyme constructs. In any embodiment the constructs can be exogenous and/or introduced by human activity.

Any of the recombinant algal cells or organisms described herein can be generated by human action, for example, by classical mutagenesis and/or genetic engineering, but can also be produced by any feasible mutagenesis method, including but not limited to any one or more of exposure to UV light, CRISPR/Cas9, cre/lox, gamma irradiation, or chemical mutagenesis. Screening methods can be used to identify mutants having desirable characteristics (e.g., reduced chlorophyll and increased lipid and/or biomass productivity. Methods for generating mutants of algal organisms using classical mutagenesis, genetic engineering, and phenotype or genotype screening are well-known in the art.

### Photosynthetic Cell or Organism

In some embodiments the photosynthetic cells or organisms of the invention can be a recombinant microalga, or a green alga. The recombinant alga can be any eukaryotic alga or microoalga such as, but not limited to, a Chlorophyte, an Ochrophyte, or a Charophyte alga. In some embodiments the mutant alga or microalga can be a Chlorophyte alga of the taxonomic Class Chlorophyceace, or of the Class Chlorodendrophyceae, or the Class Prasinophyceace, or the Class Trebouxiophyceae, or the Class Eustigmatophyceae. In some embodiments, the mutant alga or microalga can be a member of the Class Chlorophyceace, such as a species of any one or more of the genera Asteromonas, Ankistrodesmus, Carteria, Chlamydomonas, Chlorococcum, Chlorogonium, Chrysosphaera, Dunaliella, Haematococcus, Monoraphidium, Neochloris, Oedogonium, Pelagomonas, Pleurococcus, Pyrobotrys, Scenedesmus, or Volvox. In other embodiments the mutant alga or microalga of the invention can be a member of the Order Chlorodendrales, or Chlorellales. In other embodiments, the mutant alga or microalga can be a member of the Class Chlorodendrophyceae, such as a species of any one or more of the genera Prasinocladus, Scherffelia, or Tetraselmis. In further alternative embodiments, the mutant alga or microalga can be a member of the Class Prasinophyceace, optionally a species of any one or more of the genera Ostreococcus or Micromonas. Further alternatively, the mutant can be a member of the Class Trebouxiophyceae, and optionally of the Order Chlorellales, and optionally a genera selected from any one or more of Botryococcus, Chlorella, Auxenochlorella, Heveochlorella, Marinichlorella, Oocystis, Parachlorella, Pseudochlorella, Tetrachlorella, Eremosphaera, Franceia, Micractinium, Nannochloris, Picochlorum, Prototheca, Stichococcus, or Viridiella, or any of all possible combinations or sub-combination of the genera. In another embodiment the recombinant alga can be a Chlorophyte alga of the Class Trebouxiophyceae and the family Coccomyxaceae, and the genus Coccomyxa (e.g., Coccomyxa subellipsoidea). Or of the family Chlamydomonadaceae and the genus Chlamydomonas (e.g., Chlamydomonas reinhardtii); or of the family Volvocaceae and the genus Volvox (e.g., Volvox carteri, Volvox aureus, Volvox globator).

In another embodiment the mutant or recombinant alga is a Chlorophyte alga of the Class Trebouxiophyceae, or Eustigmatophyceae, and can be of the Order Chlorellales or Chlorodendrales, and can be of the Family Oocystaceae, or Chlorellaceae, or Monodopsidaceae, and optionally from a genus selected from one or more of Oocystis, Parachlorella, Picochlorum, Nannochloropsis, and Tetraselmis. The mutant or recombinant alga can also be from the genus Oocystis, or the genus Parachlorella, or the genus Picochlorum, or the genus Tetraselmis, or from any of all possible combinations and sub-combinations of the genera. In one embodiment the mutant or recombinant algal cell or organism is of the Class Trebouxiophyceae, of the Order Chlorellales, and optionally of the family Oocystaceae, and optionally can be of the genus Oocystis.

### Genetic Modification

A “genetic modification” can be any one or more of a mutation, a disruption or gene “knock out,” a deletion, an insertion, insertion of a stop codon, an inactivation, an attenuation, a rearrangement, one or more point mutations, a frameshift mutation, a nonsense mutation, an inversion, a single nucleotide polymorphism (SNP), a truncation, a point mutation, that changes the activity or expression of one or more genes or nucleic acids. In some embodiments the change in expression is a reduction or elimination of the expression or activity. The genetic modification can be made or be present in any sequence that affects expression or activity of the gene or nucleic acid sequence or the nature or quantity of its product, for example to a coding or non-coding sequence, a promoter, a terminator, an exon, an intron, a 3′ or 5′ UTR, or other regulatory sequence; a genetic modification performed in any structure of the gene can result in attenuation or elimination of the gene or nucleic acid product or activity. In some embodiments the genetic modification is a deletion, disruption, or inactivation. The genetic modification can be made to a host cell's native genome. In some embodiments, a recombinant cell or organism having attenuated expression of a gene as disclosed herein can have one or more mutations, which can be one or more nucleobase changes and/or one or more nucleobase deletions and/or one or more nucleobase insertions, into the region of a gene 5′ of the transcriptional start site, such as, in non-limiting examples, within about 2 kb, within about 1.5 kb, within about 1 kb, or within about 0.5 kb of the known or putative transcriptional start site, or within about 3 kb, within about 2.5 kb, within about 2 kb, within about 1.5 kb, within about 1 kb, or within about 0.5 kb of the translational start site.

An “attenuation” is a genetic modification resulting in a reduction of the function, activity, or expression of a gene or nucleic acid sequence compared to a corresponding (control) cell or organism not having the genetic modification being examined, i.e., the diminished function, activity, or expression is due to the genetic modification. The activity of a nucleic acid sequence can be expression of an encoded product, a binding activity (e.g., RNA binding), or other activity the nucleic acid sequence exerts within the organism. In various embodiments an attenuated gene or nucleic acid sequence produces less than 90%, or less than 80%, or less than 70%, or less than 50%, or less than 30%, or less than 20%, or less than 10%, or less than 5% or less than 1% of its function, activity, or expression of the gene or nucleic acid sequence compared to the corresponding (control) cell or organism. In various embodiments a gene attenuation can be achieved via a deletion, a disruption, or an inactivation. Any of the genetic modifications described herein can result in partial or complete attenuation of the function, activity, or expression of the attenuated gene or nucleic acid sequence.

An unmodified gene or nucleic acid sequence present naturally in the organism denotes a natural, endogenous, or wild type sequence. A deletion can mean that at least part of the object nucleic acid sequence is deleted, or that the entire sequence is deleted.

A disruption (or “knock out”) is a genetic modification that removes at least so much of the function, activity, or expression of a gene or nucleic acid sequence that any remaining function, activity, or expression of the gene or nucleic acid sequence has no significant effect on the cell or organism compared to a corresponding (control) cell or organism not having the disruption and cultivated under the same or substantially the same conditions. A deletion, disruption or “knock out”, or inactivation can also remove all function, activity, or expression of a gene or nucleic acid sequence. A “disruption” (or “knock out”) of a gene can be performed in various ways, e.g., by insertion or deletion of a nucleotide sequence into or from the coding, non-coding, or regulatory portion of a gene with resulting loss of function, activity, or expression of the gene; the loss can be such that the remaining function, activity, or expression of the gene is eliminated, or at least has no significant effect on lipid or biomass productivity. In other embodiments a disruption (or “knock out”) can involve the insertion of a stop codon, or a modification that causes a frame shift mutation. In one embodiment, a disruption can be performed by effecting a single or multi-nucleotide polymorphism into the coding, non-coding, or regulatory portion of the gene, which can result in transcription of an inactive or non-functional protein. An “inactivation” causes loss of activity or expression of an inactivated gene or nucleic acid sequence. An “inactivation” can be reversible or irreversible (for example the reversible or irreversible binding of a component to the gene or nucleic acid sequence). Thus, deletions, disruptions, and inactivations can also be attenuations. An attenuation can also be a downregulation of a gene or nucleic acid sequence, which refers to the cell or organism decreasing the amount of function, activity, or expression. Any of these genetic modifications can be introduced by standard genome modification methods (e.g., CRISPRCas9 or other standard methods). Thus, various types of genetic modifications can be given terms that overlap in description. Persons of ordinary skill know that the particular term describing a genetic modification can be dependent both on how a gene or its components, or nucleic acid sequence is being physically changed as well as on the context. The recombinant cells or organisms of the invention can have any of the types of genetic modifications described herein.

In one embodiment the genetic modification is a disruption, which in one embodiment involves the introduction of a stop codon into a gene (including regulatory sequences, e.g., a promoter), or nucleic acid sequence encoding a cryptochrome/photolyase FAD-binding domain described herein. In one embodiment the genetic modification can be a stop mutation introduced anywhere into any one of SEQ ID NOs: 2, 6, 8, or 10 (coding sequences and genomic DNA sequences of cryptochrome/photolyase FAD-binding domain from Oocystis sp.) or into a variant of either, or into a nucleic acid sequence encoding the polypeptide of any of SEQ ID NO: 1, 5, 7, or 9 (cryptochrome/photolyase FAD-binding domain polypeptide sequence in Oocystis sp.), or into a variant thereof, or into a regulatory sequence of SEQ ID NO: 2, 6, 8, or 10.

Variant sequences have at least 80% or at least 85% or at least 90% or at least 95% or at least 98% sequence identity to any nucleotide or polypeptide sequence to the reference sequence, which can be any of SEQ ID NOs: 1, 5, 7, or 9 or SEQ ID NO: 2, 6, 8, or 10, or any sequence described herein.

In other embodiments the genetic modification can be a stop mutation, nonsense mutation, or frameshift mutation introduced into a gene or nucleic acid sequence encoding a cryptochrome/photolyase FAD-binding domain disclosed herein. In various embodiments the gene or nucleic acid sequence comprises a sequence of SEQ ID NO: 2, 6, 8, or 10 (or a variant of any) or a gene or nucleic acid sequence that encodes the polypeptide of SEQ ID NO: 1, 5, 7, or 9, or a variant of any. The stop mutation, nonsense mutation, or frame shift mutation can be introduced at any location of the sequence or into a regulatory sequence governing the sequence, where the modification results in a termination of transcription from the gene prior to its natural point and disruption of the gene. Thus, in one embodiment the mutation is the introduction of a stop codon that deletes at least a portion of the gene or nucleic acid sequence, or disrupts the gene or nucleic acid sequence, and reduces or eliminates its activity or expression. The stop codon, nonsense mutation, frame-shift mutation, or other modification can also be introduced at many different loci or locations within a gene encoding a cryptochrome/photolyase FAD-binding domain, or in a regulatory sequence, for example at a promoter, terminator, or other regulatory sequence that disrupts the gene and/or reduces or eliminates the activity of the encoded polypeptide. Such insertion or deletion or other modification can also cause a loss of function or activity in the cryptochrome/photolyase FAD-binding domain and result in the effect of increased lipid productivity and/or increase biomass productivity.

Any of the recombinant cells or organisms of the invention can have a reduced functional absorption cross section of PSII and/or reduced PSII antenna size. For example, the cross-sectional unit size of the PSII antenna can be reduced by at least about 10%, at least 20%, at least 30%, at least 25%, at least 30%, at least 35%, at least 40%, at least 45%, at least 50%, at least 60%, at least about 70%, or at least about 80% compared to the functional absorption cross section of PSII and/or PSII antenna size of the corresponding (control) cell or organism not having the genetic modification. In some embodiments the recombinant cells or organisms of the invention can additionally (and optionally) have a reduced functional absorption cross section of PSI or reduced PSI antenna size by the same amounts stated above versus a corresponding (control) cell or organism.

In some embodiments, a photosynthetic cell or organism as provided herein can have increased Fv/Fm with respect to a corresponding control cell or organism. For example, the mutant photosynthetic organism may have Fv/Fm increased by at least 5%, at least 10%, at least 12%, at least 15%, at least 20%, at least 30%, at least 40% or at least 50% compared to a corresponding (control) photosynthetic organism. In various embodiments the Fv/Fm can be increased by 5-50%, or by 5-30% or by 5-20% with respect to a control photosynthetic organism.

Further, a mutant photosynthetic organism as provided herein can have an increased rate of electron transport on the acceptor side of photosystem II with respect to a control or wild type cell. The rate can be at least about 20%, 30%, 40%, 50%, 60%, 80%, or 100% higher compared to a corresponding control cell or organism. In addition, mutant photosynthetic cells or organisms of the invention can have a rate of carbon fixation (Pmax (C)) in a recombinant cell or organism as provided herein can be elevated with respect to a control organism. For example, Pmax (14C) can be increased by at least about 20%, 30%, 40%, 50%, 60%, 80%, or 100% compared to a corresponding control cell or organism.

In some embodiments, the recombinant cells or organisms of the invention have decreased PSI and/or PSII antenna size and can optionally also have a higher amount of a ribulose bisphosphate carboxylase activase (Rubisco activase or “RA”) than a corresponding (control) or wild type organism, for example, at least 1.2, 1.4, 1.6, 1.8, 2, 2.2 or 2.5, fold the amount of RA as a control organism. In some embodiments, the mutants demonstrate reduced expression of 6, 8, 10, 12, or 14 LHCP genes and increased expression of an RA gene, such as an RA-a or RA-P gene. Thus, the recombinant cells or organisms of the invention can be mutant photosynthetic organisms having reduced chlorophyll and reduced PSII antenna size where the mutants have a higher amount of Rubisco activase than control photosynthetic organisms.

The structure of a gene consists of many elements, of which the protein coding sequence is only one part. The gene includes nucleic acid sequences that are not transcribed and sequences that are untranslated regions of the RNA. Genes also contain regulatory sequences, which includes promoters, terminators, enhancers, silencers, introns, 3′ and 5′ UTRs, and coding sequences, as well as other sequences known to be a part of genes. In various embodiments any one or more of the genetic modifications described herein can be performed in any one or more of these structures or nucleic acid sequences and achieve the higher lipid productivity and/or higher biomass productivity as described herein.

The photosynthetic cells or organisms of the invention can have a higher growth rate and/or a higher biomass productivity and/or higher lipid productivity than a corresponding control cell or organism not having the genetic modification, for example, higher biomass or lipid productivity per hour or per day or per period of any one of 2 days or 3 days or 4 days or 5 days or 6 days. “Biomass” refers to cellular mass, whether of living or dead cells. Biomass productivity, or biomass accumulation, or growth rate, can be measured by any means accepted in the art, for example as ash free dry weight (AFDW), dry weight, wet weight, or total organic carbon (TOC) productivity. In any embodiment biomass productivity, or biomass accumulation, or the growth rate, can be measured as total organic carbon (TOC) productivity.

In some embodiments the photosynthetic cells or organisms of the invention can produce a greater amount of lipid and/or biomass per time period (e.g., per minute or per hour or per day or per period of 2 days or 3 days or 4 days or 5 days or 6 days), for example the biomass can be a lipid product (which can optionally be measured as FAME) than a corresponding (control) organism not having the genetic modification(s). The amount of product can be expressed in any convenient unit such as, for example, g/time period, mg/time period, ug/time period, or any other defined quantity per defined time period described herein. Such bioproducts can be isolated from a lysate or biomass or cellular secretion of any of the recombinant cells or organisms of the invention. In various embodiments, the recombinant cells or organisms of the invention produce at least 10%, at least 15%, at least 20%, at least 25%, at least 30%, at least 35%, at least 40%, at least 45%, at least 50%, at least 60%, at least 70%, at least 80%, at least 90% or at least 100% or at least 200% more of a lipid or other bioproduct than a corresponding control alga cultured under the substantially the same conditions, which can be batch, semi-continuous, or continuous culture conditions and may be nutrient replete culture conditions or may be nitrogen deplete/deficient conditions, and may be photoautotrophic conditions. Continuous culture refers to a culture of continuous nutrient replenishment, and semi-continuous culture refers to nutrient replenishment once per day, both of which are through removal of culture and resupply.

### Increased Lipid Productivity

The photosynthetic cells or organisms of the invention having a genetic modification to a gene or nucleic acid sequence encoding a cryptochrome/photolyase FAD-binding domain as described herein can demonstrate an increase in the production of lipid in the cell or organism versus a corresponding (control) cell or organism. The increase in lipid production can be measured by any accepted and suitable method, for example using fatty acid methyl ester (FAME) analysis. In one embodiment the increase in lipid production is measured as an increase in total FAME produced by the recombinant organisms. The photosynthetic cells or organisms of the invention having a genetic modification to a gene or nucleic acid encoding a cryptochrome/photolyase FAD-binding domain can exhibit at least 15% or at least 20% or at least 30% or at least 35%, or at least 40% or at least 50% or at least 60% or at least 70% or at least 80% or at least 90% or at least 100% higher lipid productivity compared to a corresponding control cell or organism, as described herein. The increase in lipid productivity versus the control organism can be measured in any convenient timeframe, e.g., after 2 days, or after 3 days, or after 4 days, or after 7 days. In other embodiments the increase in lipid productivity can be 15-25% or 15-35% or 15-45% or 15-50% or 25-45% or 25-55% or 25-70% or 25-90% or 25-100% or 25-150%, or 25-200%, or 30-35%, or 30-40%, or 35-40%, or 35-45% or 30-50% or 30-60%, which can be measured after the same timeframes as above. In one embodiment the increase can be measured weight for weight (w/w). In one embodiment lipid productivity is measured using the FAME profile (fatty acid methyl ester assay) of the respective cells or organisms. In one embodiment lipid productivity can be expressed as mg/L. In other embodiments the photosynthetic cells or organisms of the invention can exhibit at least 50 g/m² or at least 60 or at least 70 or at least 80 grams per square meter of FAME accumulation after 5 days of cultivation. Methods of producing a FAME profile are known to persons of ordinary skill in the art. A FAME profile can be determined using any suitable and accepted method, for example a method accepted by most persons of ordinary skill in the art. The photosynthetic cells or organisms of the invention can, optionally, also have an increase in biomass productivity can be 15-35% or 15-40% or 25-45% or 15-50% or 25-70% or 50-100% or 50-200% (w/w).

An increase in lipid production or lipid productivity can be measured by weight, but can also be measured in grams per square meter per day of the surface of a cultivation vessel (e.g., a flask, photobioreactor, cultivation pond). In various embodiments the photosynthetic cells or organisms of the invention produce at least 3 or at least 4 or at least 5 or at least 6 or at least 7 or at least 8 or at least 10 or at least 12 or at least 13 or at least 14 grams per square meter per day of lipid production, which can be measured by the FAME profile. In any of the embodiments the high lipid and/or high biomass productivity phenotype can be obtained under nitrogen deplete conditions, which in some embodiments can involve dilution and/or replacement of medium with fresh nitrogen deplete medium during growth. Dilutions can be by any suitable amount, for example dilution by about 50% or by about 60% or by about 70% or at least 70%, or by about 80%, or by more than 80%. In one embodiment the lipid product is a fatty acid and/or derivative of a fatty acid. In one embodiment the fatty acids and/or derivatives of fatty acid comprise one or more species of molecules having a carbon chain between C8-C18 and/or C8-C20 and/or C8-C22 and/or C8-C24, in all possible combinations and sub-combinations. In one embodiment the growth conditions can be batch growth, involving spinning cells to remove nitrogen from the medium, replacing with nitrogen deplete medium, and resuming batch growth.

In any of the embodiments the genetic modification to the gene or nucleic acid sequence encoding the cryptochrome/photolyase FAD-binding domain can result in an attenuation of expression of the respective genes. The genetic modification can be any of those described herein. In one embodiment the genetic modification is a deletion, disruption, or inactivation. In another embodiment the genetic modification is a disruption of the gene.

### Biomass Productivity

The photosynthetic cells or organisms of the invention having a genetic modification to a gene or nucleic acid encoding a cryptochrome/photolyase FAD-binding domain described herein can also have higher biomass productivity than a corresponding (control) organism not having the genetic modification. Biomass can be measured using the total organic carbon (TOC) analysis, known to persons of ordinary skill in the art. The recombinant cells can have at least 5% higher, or at least 10% higher, or at least 20% higher or at least 25% higher or at least 30% higher or at least 35% higher, or at least 50% higher or at least 60% higher or at least 70% higher or at least 80% higher or at least 90% higher or at least 100% higher or at least 125% higher or at least 150% higher or at least 200% higher biomass productivity than a corresponding (control) cell or organism, which in one embodiment can be measured by total organic carbon analysis. In other embodiments the biomass productivity can be 5-10%, or 5-15% or 10-20%, or 10-30%, or 15-35% or 15-40% or 25-45% or 15-50% or 25-70% or 50-100% or 50-200% higher than a corresponding (control) cell or organism. In any embodiment the increase in biomass productivity can be measured in any convenient timeframe, e.g., after 1 day or 2 days or 3 days or 4 days or 5 days or 7 days.

Various methods of measuring total organic carbon are known to persons of ordinary skill in the art. Biomass productivity can be measured as mg/ml of culture per time period. In some embodiments the higher biomass productivity and/or higher lipid productivity as described herein can occur under nitrogen deplete conditions. Thus, in one embodiment the recombinant alga of the invention can have higher lipid production and/or higher total organic carbon production than a corresponding (control) cell or organism, which higher amount can be produced under nitrogen deplete or low nitrogen conditions. Nitrogen deplete conditions can involve culturing in a buffer having less than 0.5 mM of nitrogen in any available form external to the cell or organism. In one embodiment the cells can be cultured in 0.5 mM or less of KNO3 or urea as a nitrogen source. Other buffers may also be used and be nitrogen deplete if they contain a level of nitrogen that does not change the physiology of a nitrogen-related parameter (e.g., lipid productivity or biomass productivity) by more than 10% versus culturing the cell in a medium free of a nitrogen source external to the cells or organisms. In any embodiment biomass productivity can be evaluated by measuring an increase in the total organic carbon of the cells. Nutrient replete conditions are those where the growth of the cultivated organism is not limited by a lack of any nutrient.

In various embodiments the one or more genetic modification(s) can be made in (i.e., derived from) a cell or organism that is a wild type, parent strain, or laboratory strain. Laboratory strains are cells or organisms that have been cultured in a laboratory setting for a period of time sufficient for the strain to undergo some adaptation(s) advantageous to growth in the laboratory environment and render the strain distinctive versus a more recently cultured wild-type strain. Laboratory strains nevertheless can be genetically modified as described herein and yield significant desirable characteristics from the genetic modification(s), as described herein. For example, laboratory strains can have higher biomass productivity and/or higher lipid productivity than a wild-type strain. In some embodiments one or more genetic modifications disclosed herein can be performed on a laboratory strain to result in a recombinant photosynthetic cell or organism of the invention. In such embodiments the laboratory strain can therefore be a corresponding control photosynthetic cell or organism described herein that does not have the genetic modification being considered.

### Carbon Partitioning

A convenient way of measuring carbon partitioning is to consider the FAME/TOC ratio. In various embodiments the recombinant photosynthetic cells or organisms can have a FAME/TOC ratio that is at least 10%, or at least 20%, or about 25% higher than a corresponding, control cell or organism cultivated under the same conditions. In any embodiment the difference in FAME/TOC ratio can be measured over any convenient period, e.g., 2 days or 3 days or 4 days or 7 days of cultivation.

### Methods of Producing Lipid

The invention also provides methods for producing a lipid-containing product. The methods involve performing a genetic modification to a photosynthetic cell or organism to produce a recombinant photosynthetic organism described herein and culturing the recombinant photosynthetic cell or organism to thereby produce a composition containing a lipid product. Any of the methods can also involve a step of harvesting lipid produced by the recombinant photosynthetic cell or organism. The culturing can be for a suitable period of time, for example, at least 1 day or at least 3 days or at least 5 days.

The invention also provides methods for producing a composition containing lipids. The methods involve culturing a recombinant photosynthetic cell or organism described herein to thereby produce a composition containing lipids. The composition can be a biomass composition. The cultivating can be done in any suitable medium conducive to algal growth (e.g., an algal growth medium or any medium described herein). The methods can also involve a step of harvesting lipids from the composition or biomass containing lipids. The methods can involve a step of harvesting lipids from the recombinant cells or organisms. Any of the methods herein can also involve a step of purifying the lipid containing composition to produce a biofuel or biofuel precursor. A biofuel precursor is a composition containing lipid molecules that can be purified into a biofuel.

The methods of producing a recombinant photosynthetic cell or organism having higher lipid productivity than a corresponding control cell or organism can involve a step of exposing photosynthetic cells or organisms to ultraviolet light to produce a photosynthetic cell or organism described herein that has higher lipid productivity than a corresponding control cell or organism. In one embodiment photosynthetic cells or organisms having higher lipid productivity can be identified by contacting the cells or organism with a stain that identifies lipids (e.g., by BODIPY dye). Optionally methods can include a step of isolating lipids from the photosynthetic cells or organisms. The photosynthetic cells or organisms can be cultivated in any suitable growth media, such as any of those described herein. In some embodiments of the methods the culture can be subjected to uv light and/or gamma radiation for a suitable period of time or under a suitable regimen. Persons of ordinary skill understand suitable regimens for uv and/or gamma radiation exposure for mutagenesis. The uv and/or gamma radiation regimen can involve exposing the cells or organisms to uv and/or gamma radiation, which can be performed in batches with each batch receiving a dose. Multiple cell batches can receive different doses of energy for each batch of cells. For example 4 or 5 batches of cells can receive doses of exposure to 16-57 uJ/cm2 of energy, and exposure energy can increase with each separate batch. The cell batches can be pooled together after exposures are complete. The photosynthetic cells or organisms (or pooled cells or organisms) can be cultivated for at least 2 days or at least 3 days, or at least 4 days, or at least 5 days, or at least 6 days, or at least 10 days, or at least 20 days, or from 2-10 days, or from 2-20 days or from 2-25 days after exposure. The photosynthetic cells or organisms can be any described herein.

Any of the photosynthetic cells or organisms of the invention can be cultivated in batch, semi-continuous, or continuous culture to produce a photosynthetic cell or organism having the higher biomass productivity and/or higher lipid productivity. In some embodiments the culture medium can be nutrient replete, or nitrogen deplete (—N). In some embodiment the culturing is under photoautotrophic conditions, and under these conditions inorganic carbon (e.g., carbon dioxide or carbonate) can be the sole or substantially the sole carbon source in the culture medium.

The invention also provides a biofuel comprising a lipid product produced by any of the recombinant cells or organisms described herein. The biofuel is produced by purifying a lipid containing composition produced by a photosynthetic cell or organism described herein.

The methods disclosed herein can product a biomass product containing a photosynthetic cell or organism described herein.

### FAME and TOC Analysis Methods

The lipid productivity of the cells or organisms can be measured by any method accepted in the art, and in one embodiment as an increase or decrease in fatty acid methyl esters comprised in the cell, i.e. FAME analysis. In some embodiments any of the recombinant algal cells or organisms of the invention can have higher biomass productivity as described herein versus corresponding control cells or organisms. In some embodiments the recombinant algal cells or organisms of the invention can have higher lipid productivity and also higher biomass productivity compared to a corresponding control cell or organism. Biomass productivity can be measured by any methods accepted in the art, for example by measuring the total organic carbon (TOC) content of a cell. Embodiments of both methods are provided in the Examples.

“FAME lipids” or “FAME” refers to lipids having acyl moieties that can be derivatized to fatty acid methyl esters, such as, for example, monoacylglycerides, diacylglycerides, triacylglycerides, wax esters, and membrane lipids such as phospholipids, galactolipids, etc. In some embodiments lipid productivity is assessed as FAME productivity in milligrams per liter (mg/L), and for algae, may be reported as grams per square meter per day (g/m2/day). In semi-continuous assays, mg/L values are converted to g/m2/day by taking into account the area of incident irradiance (the SCPA flask rack aperture of 1½ inches×33/8″, or 0.003145 m2) and the volume of the culture (550 ml). To obtain productivity values in g/m2/day, mg/L values are multiplied by the daily dilution rate (30%) and a conversion factor of 0.175. Where lipid or subcategories thereof (for example, TAG or FAME) are referred to as a percentage, the percentage is a weight percent unless indicated otherwise. The term “fatty acid product” includes free fatty acids, mono-di, or tri-glycerides, fatty aldehydes, fatty alcohols, fatty acid esters (including, but not limited to, wax esters); and hydrocarbons, including, but not limited to, alkanes and alkenes).

In some embodiments the recombinant algal organisms of the invention can have a higher FAME/TOC ratio than a corresponding control organism. In various embodiments the FAME/TOC ratio of the recombinant algal organisms of the invention can be at least 0.4 after two days of cultivation, which cultivation can be batch, continuous, or semi-continuous.

### Embodiments

In one embodiment the invention provides a photosynthetic cell or organism of the Class Trebouxiophyceae having a genetic modification in a gene or nucleic acid sequence encoding a cryptochrome/photolyase FAD-binding domain described herein. The recombinant alga exhibits higher lipid productivity and/or biomass productivity versus a corresponding control algal cell not having the genetic modification. In various embodiment the Trebouxiophyceae organism can be from the family Oocystaceae or Chlorellaceae. In one embodiment the organism is of the genus Oocystis.

In one embodiment the invention provides a recombinant Trebouxiophyceae algal organism having a deletion, disruption, or inactivation in a gene or nucleic acid sequence encoding a cryptochrome/photolyase FAD-binding domain described herein. In one embodiment the deletion, disruption, or inactivation involves the insertion of a nonsense mutation, stop mutation, or frame-shift mutation in a gene or nucleic acid sequence encoding a cryptochrome/photolyase FAD-binding domain. The cryptochrome/photolyase FAD-binding domain can be encoded by a nucleic acid sequence having at least 80%, or at least 85% or at least 90% or at least 95% sequence identity to SEQ ID NO: 2, 6, 8, or 10; and/or the encoded cryptochrome/photolyase FAD-binding domain can have at least 80%, or at least 85% or at least 90% or at least 95% sequence identity to a polypeptide selected from any of SEQ ID NO: 1, 5, 7, or 9. The recombinant alga exhibits higher lipid productivity and/or biomass productivity versus a corresponding control algal cell not having the genetic modification. The alga can be a Trebouxiophyceae organism from the family Oocystaceae, for example of the genus Oocystis. The increase in lipid productivity can be an increase of at least 30% w/w, or at least 35%, or about 39%, or 35-40% or 35-45% or 30-40%, which can be measured over a period of 2 days, or 3 days, or 4 days or 5 days or 6 days or 7 days. The recombinant cells or organisms can, optionally, also have an increase in biomass productivity versus a control cell or organism of at least 5% or at least 10% or at least 15% or at least 18% or at least 20% or at least 25%, or 5-10% or 7-10% or 10-20%, which can be measured over a period of 2 days, or 3 days, or 4 days or 5 days or 6 days or 7 days. The increase in FAME/TOC ratio can be at least 20% or 20-25% or 23-25% in 2 days.

Thus in one embodiment the recombinant cells or organisms have an increase in lipid productivity of about 30%, or at least 30%, or at least 35%; and an increase in biomass productivity of at least 8%, or 5-10%; and an increase in FAME/TOC ratio of at least 20% or 20-25% or 22-25% versus a control organism over a period of cultivation of, for example, 2 days. In another embodiment the increase in lipid productivity can be 35-40% and the increase in biomass productivity can be at least 7% or at least 8%, or 5-10% (e.g., over a period of cultivation of 2 days). In another embodiment the increase in lipid productivity can be 25-35% or 29-31%, and the increase in biomass productivity can be about 10%, or at least 8%. In another embodiment the increase in lipid productivity can be at least 30%, and the increase in biomass productivity can be at least 10% or about 10%. The increases in lipid and/or biomass productivity described can be measured over any convenient time period, in one embodiment after 2 days of cultivation.

In one embodiment the invention provides a recombinant Trebouxiophyceae organism having a deletion, disruption, or inactivation in a gene or nucleic acid sequence encoding a cryptochrome/photolyase FAD-binding domain described herein. In one embodiment the deletion, disruption, or inactivation involves the insertion of a nonsense mutation in a gene or nucleic acid sequence encoding the cryptochrome/photolyase FAD-binding domain. In one embodiment the nucleotide sequence encoding the cryptochrome/photolyase FAD-binding domain can have at least 80% or at least 90% or at least 95% sequence identity to any one or SEQ ID NO: 2, 6, 8, or 10. In one embodiment the encoded cryptochrome/photolyase FAD-binding domain can have a polypeptide sequence having at least 80% or at least 90% or at least 95% sequence identity to SEQ ID NO: 1, 5, 7, or 9. The recombinant alga exhibits higher lipid productivity and/or biomass productivity versus a corresponding control algal cell not having the genetic modification. The alga can be a Trebouxiophyceae organism from the family Oocystaceae, for example of the genus Oocystis. The increase in lipid productivity can be an increase of at least 30% or about 30%, or at least 35% w/w after 2 days of cultivation and/or an increase in biomass productivity of at least 5% or at least 7% after 2 days; and/or have an increase in the FAME/TOC ratio of at least 20% or 22-26% after 2 days. The cultivation can be done under low nitrogen or nitrogen starved conditions, in one embodiment a nitrogen level of less than 10 mg/L.

In various embodiments the cells or organisms can have an increase in lipid productivity (e.g., measured by FAME) versus a corresponding (control) cell or organism of at least 30% or at least 35% in 2 days or 3 days, or at least 35% in 7 days; and/or an increase in biomass productivity (e.g., as measured by TOC) of at least 7% or 5-10% in 2 days or 3 days, or at least 12% in 2 days or 3 days; or at least 15% in 3 days or 4 days; or at least 25% or 30% in 6 days or 7 days. The cells or organisms can also have an increase in the FAME/TOC ratio versus the corresponding (control) cell or organism of at least 20% in 2 days or 3 days; or 7-9% in 4 days or 5 days; or at least 10% or at least 12% or about 14% in 6 days or 7 days.

In other embodiments the increase in FAME areal accumulation can be at least 30% higher in 2 days, or at least 40% in 3 days, or at least 25% in 6 days, or at least 35% higher in 7 days versus the control organism. The increase in areal TOC accumulation can be at least 8% greater in 2 days or at least 15% greater in 4 days or 5 days, or at least 35% greater in 6 days or at least 25% greater in 7 days versus the control organism; and the increase in FAME/TOC ratio can be at least 20% or 20-25% or 23-25% in 2 days, or at least 14% higher in 3 days, or at least 12% higher in 7 days.

In one embodiment the invention provides a recombinant algal organism of the Class Trebouxiophyceae having a genetic modification to a gene or nucleic acid sequence encoding a cryptochrome/photolyase FAD-binding domain. In one embodiment the gene or nucleic acid sequence is that of that SEQ ID NO: 2, 6, 8, or 10, or a variant of any of them. The genetic modification can be a deletion (optionally a functional deletion) or disruption of the gene or nucleic acid sequence. The recombinant alga exhibits higher lipid productivity and, optionally, higher biomass productivity versus a corresponding control algal cell not having the genetic modification. In various embodiment the Trebouxiophyceae organism can be from the family Oocystaceae or Chlorellaceae. In one embodiment the organism is of the genus Oocystis.

### Example 1

This example illustrates the mutagenesis and screening of wild-type cells. Oocystis sp. cells (STR00015, wild-type) were acclimated to diel growth in culture flasks at a light intensity of about 100 uE and 1% CO2 in urea supplemented minimal medium for a week. The culture was scaled up for 3 days in 500 mL square-bottom flasks, bubbled with 1% CO2 at a maximum irradiance of about 1400 uE under diel conditions, to an OD730 of about 1.0. The culture was then centrifuged at 5000 g for 10 mins and the cell pellets resuspended in nitrogen-free minimal medium to an OD730 of about 0.9. This nitrogen-free culture was then incubated for 48 hrs in square-bottom flasks bubbled with 1% CO2 at a maximum irradiance of ˜1400 uE under diel conditions.

The cells were then mutagenized using uv light at a concentration of 2e6 cell/ml and at 22.4, 33.6, 44.8 and 56 mJ/cm² in a uv crosslinker apparatus. Mutagenized cells were allowed to recover in the dark for 48 hours. Cultures were scaled up in low light (about 100 uE) before enrichment.

Mutagenized cells were acclimated to diel growth in culture flasks at a light intensity of about 100 uE and 1% CO2 in urea supplemented minimal medium for a week. The culture was scaled up for 3 days 500 mL square-bottom flasks, bubbled with 1% CO2 at a maximum irradiance of about 1400 uE under diel conditions, to an OD730 of about 1.0. The culture was then centrifuged at 5000 g for 10 mins and the cell pellets resuspended in nitrogen-free minimal medium to an OD730 of about 0.9. This nitrogen-free culture was then incubated for 48 hrs under the same conditions.

After 48 hours of nitrogen-free batch growth, an aliquot of cells was removed and subjected to staining with the lipid-specific BODIPY dye for 10 minutes in the dark at a final concentration of 0.2 ug/ml. Mutant cells with the highest level of BODIPY staining were enriched by fluorescence activated cell sorting (FACS). Enriched cell populations were starved for nitrogen as above and subjected to further BODIPY-based FACS enrichment. This iterative process was repeated for a total of five rounds retaining the top cells in each round. The final cells were plated on minimal medium agar plates supplemented with urea to isolate single axenic colonies.

Isolated mutants were scaled up in tissue culture flasks in minimal medium supplemented with urea, then transitioned to nitrogen-free minimal medium. The lipid and biomass accumulation of isolated mutants were compared to the parental strain wild-type cells (Strain 15) with lipid content measured by total fatty acid methyl ester (FAME) analysis and biomass measured by total organic carbon (TOC). As shown in FIGS. 1a-c, one mutagenized strain from the screen (STR27559) showed an increase in accumulated FAME and TOC at 48 hours in nitrogen deplete minimal medium, as well as an increase in TOC accumulation and an increase in FAME/TOC ratio—an indicator of how much fixed carbon is partitioned to lipids. The results indicated that the isolated mutagenized strain STR27559 exhibited improved lipid productivity over the parental wild-type strain. Proline F/2 algae food was used as the nitrogen deplete medium and was made by adding 1.3 ml PROLINE® F/2 Algae Feed Part A (Pentair Aquatic Eco-Systems, Inc., Cary, NC) and 1.3 ml ‘Solution C’ to a final volume of 1 liter of a solution of aquarium salts (17.5 g/L). Solution C is 38.75 g/L NaH2PO4H2O, 758 mg/L Thiamine HCl, 3.88 mg/L vitamin B12, and 3.84 mg/L biotin. However, persons of ordinary skill in the art with reference to the present disclosure will realize that many algae foods or media can be used with the nitrogen content minimized, such as by omitting urea or available nitrates.

### Example 2

Genomic DNA was extracted from wild-type (STR00015) and mutagenized STR27559 cells and sequenced on an automated sequencer. Paired end reads of 150 bp were processed and mapped to a reference genome previously generated for the wild-type cell. Small variant detection as conducted for both STR00015 and STR27559 using small-variant detection software. Small variant analysis revealed 543 unique variants in STR27559 compared to the wild-type.

A list of the mutations identified within exons or at splice junctions in the mutated strain (STR27559) are set out in FIG. 2. To identify which mutation(s) cause the high lipid phenotype independent knockouts of genes bearing SNPs in the mutated strain were conducted via RNP-based Cas9-mediated gene disruption using biolistic transformation in the wild-type (STR00015) parental strain. All the strains generated were tested for improved biomass and lipid accumulation during nitrogen starvation in T25 flasks.

Thirty-one variants were located within or near gene exon coding sequences, all of which resulted in non-synonymous codon substitutions, and three that resulted in the introduction of a stop codon that prematurely terminates translation of the gene and truncates the translated protein (FIG. 2a). Only the three gene targets, including CRY1, with stop codon introductions were pursued for gene editing recapitulation.

In one gene encoding a cryptochrome/photolyase FAD-binding domain a single nucleotide polymorphism (SNP) was discovered at Gln56. This mutation was found to change the Gln56 codon sequence CAG to TAG, i.e. a stop codon that prematurely ends translation and results in a truncated protein and a knockout of the cryptochrome/photolyase FAD-binding domain gene product.

### Example 3

The CRISPR-Cas9 gene editing platform was used to recapitulate the mutation and generate strains with targeted modifications of the CRY1 gene. RNP-based Cas9-mediated gene editing methods were used to produce independent CRY1 knockout strains at three different loci within the CRY1 coding sequence. The target locations were determined by proximity to the STR27559 variant locus and by the sgRNA target sequence that is limited to the nucleotide sequence 5′N20NGG-3′ (FIG. 2b).

One Cas9-mediated knockout strain (STR32197) contained a targeted modification of a single guanine (G) nucleotide insertion at the target site of sgRNA-2 that caused a codon frame-shift downstream of the insertion and the introduction of a premature stop codon (see SEQ ID NO: 3 and SEQ ID NO: 4 for the protein and CDS nucleotide sequences, respectively). The CRY1 modification in STR32197 was considered deleterious to the protein function of CRY1 and thus STR32197 was deemed a CRY1 knockout strain. Notably, STR32197 was derived from parent strain STR31208, which already had knockouts of the genes YVTN (SEQ ID NO: 14) (WD40 repeat domain), RBD (SEQ ID NO: 15) (RNA binding domain), and APGL (SEQ ID NO: 16) (ADP-glucose pyrophosphorylase).

### Example 4

The STR32197 CRY1 knockout strain was analyzed for FAME and TOC accumulation to assess the impact of the CRY1 knockout genotype. Knockout strain STR32197 and its parent strain (STR31208) were grown for seven days in nitrogen starvation conditions in minimal medium and FAME and TOC accumulation and the FAME/TOC carbon partitioning was assessed each day (FIG. 3). FAME accumulation was higher in STR31297 compared to its parent STR31208 in every timeframe of nitrogen starvation (FIG. 3a). For TOC accumulation, STR32197 was higher than its parent strain every day for Days 1-7 (FIG. 3b). The FAME/TOC carbon partitioning was also improved for STR32197 for Days 1˜4 and 7 (FIG. 3c). On day seven of nitrogen starvation, FAME and TOC were improved by 37% and 28%, respectively, for STR32197 compared to the parent strain. Additionally, carbon partitioning was improved by 14% for STR32197 compared to its parent strain.

The FAME and TOC accumulation data for STR32197 indicated that the knockout genotype in the CRY1 gene was responsible for the lipid productivity improvement. This data led to the conclusion that the genetic modification in the CRY1 gene by insertion of a stop codon caused the high lipid production phenotype in the mutant strain STR27559. Thus, a knockout of 3EUKT4137726_1 (Cryptochrome/photolyase FAD-binding domain or CRY1) in an Oocystis sp. strain significantly improved lipid productivity and carbon partitioning.

Additional CRY1 knockout strains were produced to determine if the lipid productivity improvement could be translated to multiple strain backgrounds, even those having multiple other genes disrupted or knocked out. STR33125 was generated by RNP-based Cas9-mediated mutagenesis in the strain background STR31187 (already having knockouts of YVTN (WD40 repeat domains), RBD (an RNA binding domain) and AGPL (ADP-glucose pyrophosphorylase)) at the target locus determined by sgRNA-3 (FIG. 2b). The resulting modification was a single nucleotide insertion of an adenine (A) at the sgRNA-3 cut site (inserted after position 722) that resulted in a frame-shift and the introduction of a premature stop codon (at position 139 of the amino acid sequence) (see SEQ ID NOs: 9-10 for the amino acid and CDS nucleotide sequences, respectively, of STR33125). Additionally, STR32510 was generated by RNP-based Cas9-mediated mutagenesis in the strain background STR31378 (which already had knockouts of YVTN, RBD, PK1 (protein kinase of SEQ ID NO: 17) at the target locus determined by sgRNA-2 (FIG. 2b). The resulting modification was a single nucleotide insertion of a cysteine (C) at the sgRNA-1 cut site that resulted in a protein frame-shift and the introduction of a premature stop codon (SEQ ID NOs: 7-8 show the amino acid and CDS nucleotide sequences, respectively, of STR32510). The FAME and TOC accumulation phenotypes were analyzed using the same methods described above for STR32197. The relative improvements in FAME and TOC accumulation and the FAME/TOC carbon partitioning compared to the parent strains are shown in FIG. 2c for all three knockout strains.

All three CRY1 knockout strains showed improvements in FAME, TOC, and FAME/TOC compared to their parent strains. Significant improvements in lipid productivity and carbon partitioning were therefore obtained by a CRY1 knockout regardless of the background strain genotype.

### Example 5

Functional domains for CRY1 were analyzed using the wild-type amino acid sequence (SEQ ID NO: 1). The CRY1 amino acid sequence (696 amino acids in length) was submitted to publicly available protein databases for comparative analysis to known protein functional domains. Two functional domains were revealed. The first was an N-terminal DNA photolyase (IPR006050) of 170-AA length spanning the protein from amino acid position 15 to 185. The second is a Cryptochrome/FAD binding domain of DNA photolyase (IPR005101) of 197-AA length spanning the protein from amino acid position 304 to 501. The called functional domains indicate that CRY1 is a blue-light sensing protein that is potentially involved in repairing UV-damaged DNA (photolyase activity) and circadian clock maintenance (cryptochrome activity).

A BLAST search for orthologous proteins in other organisms revealed that cryptochromes are ubiquitous amongst green algae, plants, and bacteria. The closest related in terms of percent identity are green algae orthologs that include cryptochrome 1 protein (Nannochloropsis gaditana CCMP526) and Deoxyribodipyrimidine photolyase, class 1 (Volvox carteri f. nagariensis) (FIG. 4). The most studied of these orthologs is the uncharacterized protein CHLRE_06 g295200v5 (Chlamydomonas reinhardtii) that is more commonly known as CPH1. CPH1 has been characterized as a “plant-like” cryptochrome that is found in many land plants including the model flowering plants, Arabidopsis thaliana. CPH1 has been shown to display regulatory functions of circadian clock and cellular sexual cycle (Jan Petersen et al. Frontiers in Plant Science, Vol. 12, 2021, The World of Algae Reveals a Broad Variety of Cryptochrome Properties and Functions.”

Although the invention has been described with reference to the presently preferred embodiment, it should be understood that various modifications can be made without departing from the spirit of the invention. Accordingly, the invention is limited only by the following claims.

Sequence Listing
SEQ ID NO: 1-PRT, Oocystis sp., CRY1 wild type
MAMPINPYNAKHETAVVWFRRDLRISDNPALAAALQSATNVIPLFIWAPEEEGQF
QPGRANRWWLRNSLLSLSAELERLGSRLVCMAAPDCQSALCHVLASTGASALFTN
RLYDPITMVRDNELLARVAGLGVRCFMFNSDLLYEPWEVLDQAGKPFNSFDAFWG
RSPLPFSLEHAHRVTHMPHPPLPPIPAPVALPTVPGSVCGTLVDSLGLLTEEELL
TNQQLEFTWSPGSAGAHKLFAKFVSRRLRQFQHDRAKSDRNSTSRLSPHVHFGEI
SVRYMYYVVKQKEAEWLATGERITSCADFLQQMGYREYSRYLSWHFPFTHERSLL
EHVRACPYRMDQRLFKAWRQGNTGYPLVDAAMRELWSSGWMHNRMRVTAASFLVK
HLLLPWQWGLKHFWDALVDADLEADALGWQYCAGCLADAHPLDYMIDHGTESKRF
DPDGQYVRRWLPVLARVPRQYIHEPWTAPQHVLDEAGVELGSNYPWPVVDAGEAQ
VLLGAAKDVVDAALEALNETQRGPYRPPSLNPLDPSAPDSLRRMYAKFQHQLTRT
AESAASGQRPSKRTTMEAAAQQAREDMQVCGQGQGGSSDNEEVESNMGVVDALLG
GMAQLAGDDTAEVVQRRHVHRRRPSSCATGGSTQDGPLARRPAAGSGCGSAPDAP
QQDGASPDAPWQHEAPPRGNGQAKHAALQPSNAWGS*
SEQ ID NO: 2-DNA, Oocystis sp., CRY1 wild type CDS
ATGGCGATGCCGATAAACCCGTACAATGCAAAGCACGAGACAGCGGTTGTATGGT
TCAGGCGGGACTTGCGCATCTCTGATAACCCGGCTCTAGCCGCAGCGCTCCAGTC
GGCGACCAACGTGATCCCGCTGTTCATATGGGCGCCAGAGGAGGAGGGCCAGTTC
CAGCCGGGCCGTGCTAACCGATGGTGGCTGCGCAACTCGCTGCTGTCGCTGTCCG
CCGAGCTCGAGCGGCTGGGCAGCAGGCTGGTGTGCATGGCCGCGCCCGACTGCCA
GTCGGCGCTCTGCCACGTGCTGGCCAGCACCGGTGCCAGCGCGCTGTTCACCAAC
CGACTGTACGATCCCATCACCATGGTGAGGGACAACGAGCTGCTCGCACGCGTGG
CCGGCCTGGGTGTGCGCTGCTTCATGTTTAACTCCGACCTGCTGTACGAGCCATG
GGAGGTGCTGGACCAGGCGGGCAAGCCGTTCAACTCGTTCGACGCGTTTTGGGGC
AGGTCCCCTTTGCCCTTCTCACTGGAACACGCGCACAGGGTGACGCACATGCCGC
ACCCGCCCCTGCCTCCGATCCCGGCGCCCGTGGCGCTCCCCACCGTGCCTGGCAG
CGTGTGCGGCACGCTGGTCGACTCCCTTGGCCTACTGACGGAGGAGGAGCTGCTG
ACTAACCAGCAGCTGGAGTTCACGTGGTCCCCGGGCAGTGCCGGTGCGCACAAGC
TGTTTGCCAAGTTTGTGTCCAGGCGTCTTCGCCAGTTCCAGCACGATCGGGCCAA
GTCAGACCGGAACAGCACCTCGCGCCTGTCGCCGCACGTGCACTTCGGCGAGATC
AGCGTGCGGTACATGTACTACGTGGTCAAGCAGAAGGAAGCCGAGTGGCTGGCGA
CGGGAGAGCGCATCACCAGCTGTGCGGACTTCCTGCAGCAGATGGGGTACCGCGA
GTACTCGCGCTACCTCTCCTGGCACTTCCCCTTCACCCACGAGCGCTCCCTGCTT
GAGCACGTGCGCGCCTGCCCGTACCGCATGGACCAAAGGCTGTTCAAGGCTTGGC
GGCAGGGCAACACCGGGTACCCTCTGGTTGACGCGGCCATGCGAGAGCTGTGGAG
CTCCGGCTGGATGCACAACCGCATGAGGGTGACCGCTGCTTCCTTTTTGGTCAAG
CACCTGCTGCTGCCCTGGCAGTGGGGCCTGAAGCACTTTTGGGACGCGCTGGTGG
ACGCAGATCTGGAGGCCGATGCACTGGGGTGGCAGTACTGCGCAGGCTGCCTGGC
AGACGCACACCCCCTGGACTACATGATCGACCACGGGACCGAGTCCAAGCGGTTT
GACCCCGACGGCCAGTACGTGCGCCGCTGGCTGCCCGTGCTGGCAAGGGTGCCCA
GGCAATACATACACGAGCCGTGGACCGCACCGCAGCATGTGCTGGACGAGGCGGG
CGTCGAGCTCGGCTCCAACTACCCCTGGCCGGTGGTCGACGCGGGGGAGGCGCAG
GTGCTGCTGGGCGCTGCCAAGGACGTGGTGGACGCCGCCCTGGAGGCGCTCAACG
AGACGCAGAGAGGGCCCTACCGGCCGCCCAGCCTCAACCCCCTCGACCCATCCGC
GCCGGACTCGCTGCGGCGCATGTATGCCAAGTTCCAGCACCAGCTCACGCGCACG
GCGGAGTCGGCGGCCAGCGGGCAGCGGCCCTCCAAGCGGACAACGATGGAGGCAG
CAGCGCAGCAGGCACGGGAGGACATGCAGGTGTGCGGCCAGGGCCAGGGCGGCTC
CTCTGACAACGAGGAAGTCGAGAGCAACATGGGGGTGGTTGACGCGCTGCTCGGC
GGCATGGCGCAACTCGCCGGTGATGACACGGCGGAGGTGGTGCAGCGCCGCCACG
TGCACCGCCGCCGGCCGTCCTCCTGCGCAACAGGAGGCAGTACGCAGGACGGCCC
GCTCGCGCGCCGACCCGCCGCCGGCAGCGGATGCGGCAGCGCGCCAGATGCTCCA
CAGCAAGATGGGGCATCGCCAGACGCTCCTTGGCAACACGAGGCACCGCCCCGAG
GCAATGGCCAAGCAAAGCATGCGGCTCTGCAGCCAAGCAACGCATGGGGCTCTTG
A
SEQ ID NO: 3-PRT, Oocystis sp., CRY1 in
mutagenized strain 32197
MAMPINPYNAKHETAVVWFRRDLRISDNPALAAALQSATNVIPLFIWAPEEEGQF*
SEQ ID NO: 4-DNA, Oocystis sp., CRY1 CDS in
mutagenized strain 32197
ATGGCGATGCCGATAAACCCGTACAATGCAAAGCACGAGACAGCGGTTGTATGGT
TCAGGCGGGACTTGCGCATCTCTGATAACCCGGCTCTAGCCGCAGCGCTCCAGTC
GGCGACCAACGTGATCCCGCTGTTCATATGGGCGCCAGAGGAGGAGGGCCAGTTC
TAGCCGGGCCGTGCTAACCGATGGTGGCTGCGCAACTCGCTGCTGTCGCTGTCCG
CCGAGCTCGAGCGGCTGGGCAGCAGGCTGGTGTGCATGGCCGCGCCCGACTGCCA
GTCGGCGCTCTGCCACGTGCTGGCCAGCACCGGTGCCAGCGCGCTGTTCACCAAC
CGACTGTACGATCCCATCACCATGGTGAGGGACAACGAGCTGCTCGCACGCGTGG
CCGGCCTGGGTGTGCGCTGCTTCATGTTTAACTCCGACCTGCTGTACGAGCCATG
GGAGGTGCTGGACCAGGCGGGCAAGCCGTTCAACTCGTTCGACGCGTTTTGGGGC
AGGTCCCCTTTGCCCTTCTCACTGGAACACGCGCACAGGGTGACGCACATGCCGC
ACCCGCCCCTGCCTCCGATCCCGGCGCCCGTGGCGCTCCCCACCGTGCCTGGCAG
CGTGTGCGGCACGCTGGTCGACTCCCTTGGCCTACTGACGGAGGAGGAGCTGCTG
ACTAACCAGCAGCTGGAGTTCACGTGGTCCCCGGGCAGTGCCGGTGCGCACAAGC
TGTTTGCCAAGTTTGTGTCCAGGCGTCTTCGCCAGTTCCAGCACGATCGGGCCAA
GTCAGACCGGAACAGCACCTCGCGCCTGTCGCCGCACGTGCACTTCGGCGAGATC
AGCGTGCGGTACATGTACTACGTGGTCAAGCAGAAGGAAGCCGAGTGGCTGGCGA
CGGGAGAGCGCATCACCAGCTGTGCGGACTTCCTGCAGCAGATGGGGTACCGCGA
GTACTCGCGCTACCTCTCCTGGCACTTCCCCTTCACCCACGAGCGCTCCCTGCTT
GAGCACGTGCGCGCCTGCCCGTACCGCATGGACCAAAGGCTGTTCAAGGCTTGGC
GGCAGGGCAACACCGGGTACCCTCTGGTTGACGCGGCCATGCGAGAGCTGTGGAG
CTCCGGCTGGATGCACAACCGCATGAGGGTGACCGCTGCTTCCTTTTTGGTCAAG
CACCTGCTGCTGCCCTGGCAGTGGGGCCTGAAGCACTTTTGGGACGCGCTGGTGG
ACGCAGATCTGGAGGCCGATGCACTGGGGTGGCAGTACTGCGCAGGCTGCCTGGC
AGACGCACACCCCCTGGACTACATGATCGACCACGGGACCGAGTCCAAGCGGTTT
GACCCCGACGGCCAGTACGTGCGCCGCTGGCTGCCCGTGCTGGCAAGGGTGCCCA
GGCAATACATACACGAGCCGTGGACCGCACCGCAGCATGTGCTGGACGAGGCGGG
CGTCGAGCTCGGCTCCAACTACCCCTGGCCGGTGGTCGACGCGGGGGAGGCGCAG
GTGCTGCTGGGCGCTGCCAAGGACGTGGTGGACGCCGCCCTGGAGGCGCTCAACG
AGACGCAGAGAGGGCCCTACCGGCCGCCCAGCCTCAACCCCCTCGACCCATCCGC
GCCGGACTCGCTGCGGCGCATGTATGCCAAGTTCCAGCACCAGCTCACGCGCACG
GCGGAGTCGGCGGCCAGCGGGCAGCGGCCCTCCAAGCGGACAACGATGGAGGCAG
CAGCGCAGCAGGCACGGGAGGACATGCAGGTGTGCGGCCAGGGCCAGGGCGGCTC
CTCTGACAACGAGGAAGTCGAGAGCAACATGGGGGTGGTTGACGCGCTGCTCGGC
GGCATGGCGCAACTCGCCGGTGATGACACGGCGGAGGTGGTGCAGCGCCGCCACG
TGCACCGCCGCCGGCCGTCCTCCTGCGCAACAGGAGGCAGTACGCAGGACGGCCC
GCTCGCGCGCCGACCCGCCGCCGGCAGCGGATGCGGCAGCGCGCCAGATGCTCCA
CAGCAAGATGGGGCATCGCCAGACGCTCCTTGGCAACACGAGGCACCGCCCCGAG
GCAATGGCCAAGCAAAGCATGCGGCTCTGCAGCCAAGCAACGCATGGGGCTCTTG
A
SEQ ID NO: 5-PRT, Oocystis sp., CRY1 in strain 32510
MAMPINPYNAKHETAVVWFRRDLRISDNPALAAALQSATNVIPLFIWAPEEEGQF
QPGRANRWWLRNSLLSLSAELERLGSRLVCMAAPDCQSALCHVLASTGASALFTN
RLYDPITMVRDNELLARVAGLGVRCFMFNSDRAVRAMGGAGPGGQAVQLVRRVLG
QVPFALLTGTRAQGDAHAAPAPASDPGARGAPHRAWQRVRHAGRLPWPTDGGGAA
D*
SEQ ID NO: 6-DNA, Oocystis sp., CRY1 CDS in strain 32510
ATGGCGATGCCGATAAACCCGTACAATGCAAAGCACGAGACAGCGGTTGTATGGT
TCAGGCGGGACTTGCGCATCTCTGATAACCCGGCTCTAGCCGCAGCGCTCCAGTC
GGCGACCAACGTGATCCCGCTGTTCATATGGGCGCCAGAGGAGGAGGGCCAGTTC
CAGCCGGGCCGTGCTAACCGATGGTGGCTGCGCAACTCGCTGCTGTCGCTGTCCG
CCGAGCTCGAGCGGCTGGGCAGCAGGCTGGTGTGCATGGCCGCGCCCGACTGCCA
GTCGGCGCTCTGCCACGTGCTGGCCAGCACCGGTGCCAGCGCGCTGTTCACCAAC
CGACTGTACGATCCCATCACCATGGTGAGGGACAACGAGCTGCTCGCACGCGTGG
CCGGCCTGGGTGTGCGCTGCTTCATGTTTAACTCCGACCGTGCTGTACGAGCCAT
GGGAGGTGCTGGACCAGGCGGGCAAGCCGTTCAACTCGTTCGACGCGTTTTGGGG
CAGGTCCCCTTTGCCCTTCTCACTGGAACACGCGCACAGGGTGACGCACATGCCG
CACCCGCCCCTGCCTCCGATCCCGGCGCCCGTGGCGCTCCCCACCGTGCCTGGCA
GCGTGTGCGGCACGCTGGTCGACTCCCTTGGCCTACTGACGGAGGAGGAGCTGCT
GACTAACCAGCAGCTGGAGTTCACGTGGTCCCCGGGCAGTGCCGGTGCGCACAAG
CTGTTTGCCAAGTTTGTGTCCAGGCGTCTTCGCCAGTTCCAGCACGATCGGGCCA
AGTCAGACCGGAACAGCACCTCGCGCCTGTCGCCGCACGTGCACTTCGGCGAGAT
CAGCGTGCGGTACATGTACTACGTGGTCAAGCAGAAGGAAGCCGAGTGGCTGGCG
ACGGGAGAGCGCATCACCAGCTGTGCGGACTTCCTGCAGCAGATGGGGTACCGCG
AGTACTCGCGCTACCTCTCCTGGCACTTCCCCTTCACCCACGAGCGCTCCCTGCT
TGAGCACGTGCGCGCCTGCCCGTACCGCATGGACCAAAGGCTGTTCAAGGCTTGG
CGGCAGGGCAACACCGGGTACCCTCTGGTTGACGCGGCCATGCGAGAGCTGTGGA
GCTCCGGCTGGATGCACAACCGCATGAGGGTGACCGCTGCTTCCTTTTTGGTCAA
GCACCTGCTGCTGCCCTGGCAGTGGGGCCTGAAGCACTTTTGGGACGCGCTGGTG
GACGCAGATCTGGAGGCCGATGCACTGGGGTGGCAGTACTGCGCAGGCTGCCTGG
CAGACGCACACCCCCTGGACTACATGATCGACCACGGGACCGAGTCCAAGCGGTT
TGACCCCGACGGCCAGTACGTGCGCCGCTGGCTGCCCGTGCTGGCAAGGGTGCCC
AGGCAATACATACACGAGCCGTGGACCGCACCGCAGCATGTGCTGGACGAGGCGG
GCGTCGAGCTCGGCTCCAACTACCCCTGGCCGGTGGTCGACGCGGGGGAGGCGCA
GGTGCTGCTGGGCGCTGCCAAGGACGTGGTGGACGCCGCCCTGGAGGCGCTCAAC
GAGACGCAGAGAGGGCCCTACCGGCCGCCCAGCCTCAACCCCCTCGACCCATCCG
CGCCGGACTCGCTGCGGCGCATGTATGCCAAGTTCCAGCACCAGCTCACGCGCAC
GGCGGAGTCGGCGGCCAGCGGGCAGCGGCCCTCCAAGCGGACAACGATGGAGGCA
GCAGCGCAGCAGGCACGGGAGGACATGCAGGTGTGCGGCCAGGGCCAGGGCGGCT
CCTCTGACAACGAGGAAGTCGAGAGCAACATGGGGGTGGTTGACGCGCTGCTCGG
CGGCATGGCGCAACTCGCCGGTGATGACACGGCGGAGGTGGTGCAGCGCCGCCAC
GTGCACCGCCGCCGGCCGTCCTCCTGCGCAACAGGAGGCAGTACGCAGGACGGCC
CGCTCGCGCGCCGACCCGCCGCCGGCAGCGGATGCGGCAGCGCGCCAGATGCTCC
ACAGCAAGATGGGGCATCGCCAGACGCTCCTTGGCAACACGAGGCACCGCCCCGA
GGCAATGGCCAAGCAAAGCATGCGGCTCTGCAGCCAAGCAACGCATGGGGCTCTT
GA
SEQ ID NO: 7-PRT, Oocystis sp., CRY1 in strain 33510
MAMPINPYNAKHETAVVWFRRDLRISDNPALAAALQSATNVIPLLHMGARGGGPVPAG
PC*
SEQ ID NO: 8-DNA, Oocystis sp., CRY1 CDS in strain 33510
ATGGCGATGCCGATAAACCCGTACAATGCAAAGCACGAGACAGCGGTTGTATGGT
TCAGGCGGGACTTGCGCATCTCTGATAACCCGGCTCTAGCCGCAGCGCTCCAGTC
GGCGACCAACGTGATCCCGCTGTTCATATGGGCGCCAGAGGAGGAGGGCCAGTTC
CAGCCGGGCCGTGCTAACCGATGGTGGCTGCGCAACTCGCTGCTGTCGCTGTCCG
CCGAGCTCGAGCGGCTGGGCAGCAGGCTGGTGTGCATGGCCGCGCCCGACTGCCA
GTCGGCGCTCTGCCACGTGCTGGCCAGCACCGGTGCCAGCGCGCTGTTCACCAAC
CGACTGTACGATCCCATCAACCATGGTGAGGGACAACGAGCTGCTCGCACGCGTG
GCCGGCCTGGGTGTGCGCTGCTTCATGTTTAACTCCGACCTGCTGTACGAGCCAT
GGGAGGTGCTGGACCAGGCGGGCAAGCCGTTCAACTCGTTCGACGCGTTTTGGGG
CAGGTCCCCTTTGCCCTTCTCACTGGAACACGCGCACAGGGTGACGCACATGCCG
CACCCGCCCCTGCCTCCGATCCCGGCGCCCGTGGCGCTCCCCACCGTGCCTGGCA
GCGTGTGCGGCACGCTGGTCGACTCCCTTGGCCTACTGACGGAGGAGGAGCTGCT
GACTAACCAGCAGCTGGAGTTCACGTGGTCCCCGGGCAGTGCCGGTGCGCACAAG
CTGTTTGCCAAGTTTGTGTCCAGGCGTCTTCGCCAGTTCCAGCACGATCGGGCCA
AGTCAGACCGGAACAGCACCTCGCGCCTGTCGCCGCACGTGCACTTCGGCGAGAT
CAGCGTGCGGTACATGTACTACGTGGTCAAGCAGAAGGAAGCCGAGTGGCTGGCG
ACGGGAGAGCGCATCACCAGCTGTGCGGACTTCCTGCAGCAGATGGGGTACCGCG
AGTACTCGCGCTACCTCTCCTGGCACTTCCCCTTCACCCACGAGCGCTCCCTGCT
TGAGCACGTGCGCGCCTGCCCGTACCGCATGGACCAAAGGCTGTTCAAGGCTTGG
CGGCAGGGCAACACCGGGTACCCTCTGGTTGACGCGGCCATGCGAGAGCTGTGGA
GCTCCGGCTGGATGCACAACCGCATGAGGGTGACCGCTGCTTCCTTTTTGGTCAA
GCACCTGCTGCTGCCCTGGCAGTGGGGCCTGAAGCACTTTTGGGACGCGCTGGTG
GACGCAGATCTGGAGGCCGATGCACTGGGGTGGCAGTACTGCGCAGGCTGCCTGG
CAGACGCACACCCCCTGGACTACATGATCGACCACGGGACCGAGTCCAAGCGGTT
TGACCCCGACGGCCAGTACGTGCGCCGCTGGCTGCCCGTGCTGGCAAGGGTGCCC
AGGCAATACATACACGAGCCGTGGACCGCACCGCAGCATGTGCTGGACGAGGCGG
GCGTCGAGCTCGGCTCCAACTACCCCTGGCCGGTGGTCGACGCGGGGGAGGCGCA
GGTGCTGCTGGGCGCTGCCAAGGACGTGGTGGACGCCGCCCTGGAGGCGCTCAAC
GAGACGCAGAGAGGGCCCTACCGGCCGCCCAGCCTCAACCCCCTCGACCCATCCG
CGCCGGACTCGCTGCGGCGCATGTATGCCAAGTTCCAGCACCAGCTCACGCGCAC
GGCGGAGTCGGCGGCCAGCGGGCAGCGGCCCTCCAAGCGGACAACGATGGAGGCA
GCAGCGCAGCAGGCACGGGAGGACATGCAGGTGTGCGGCCAGGGCCAGGGCGGCT
CCTCTGACAACGAGGAAGTCGAGAGCAACATGGGGGTGGTTGACGCGCTGCTCGG
CGGCATGGCGCAACTCGCCGGTGATGACACGGCGGAGGTGGTGCAGCGCCGCCAC
GTGCACCGCCGCCGGCCGTCCTCCTGCGCAACAGGAGGCAGTACGCAGGACGGCC
CGCTCGCGCGCCGACCCGCCGCCGGCAGCGGATGCGGCAGCGCGCCAGATGCTCC
ACAGCAAGATGGGGCATCGCCAGACGCTCCTTGGCAACACGAGGCACCGCCCCGA
GGCAATGGCCAAGCAAAGCATGCGGCTCTGCAGCCAAGCAACGCATGGGGCTCTT
GA
SEQ ID NO: 9-PRT, Oocystis sp., CRY1 protein, Strain 33125
MAMPINPYNAKHETAVVWFRRDLRISDNPALAAALQSATNVIPLFIWAPEEEGQF
QPGRANRWWLRNSLLSLSAELERLGSRLVCMAAPDCQSALCHVLASTGASALFTN
RLYDPINHGEGQRAARTRGRPGCALLHV*
SEQ ID NO: 10-DNA, Oocystis sp., CRY1 CDS, Strain 33125
ATGGCGATGCCGATAAACCCGTACAATGCAAAGCACGAGACAGCGGTTGTATGGT
TCAGGCGGGACTTGCGCATCTCTGATAACCCGGCTCTAGCCGCAGCGCTCCAGTC
GGCGACCAACGTGATCCCGCTGTTCATATGGGCGCCAGAGGAGGAGGGCCAGTTC
CAGCCGGGCCGTGCTAACCGATGGTGGCTGCGCAACTCGCTGCTGTCGCTGTCCG
CCGAGCTCGAGCGGCTGGGCAGCAGGCTGGTGTGCATGGCCGCGCCCGACTGCCA
GTCGGCGCTCTGCCACGTGCTGGCCAGCACCGGTGCCAGCGCGCTGTTCACCAAC
CGACTGTACGATCCCATCAACCATGGTGAGGGACAACGAGCTGCTCGCACGCGTG
GCCGGCCTGGGTGTGCGCTGCTTCATGTTTAACTCCGACCTGCTGTACGAGCCAT
GGGAGGTGCTGGACCAGGCGGGCAAGCCGTTCAACTCGTTCGACGCGTTTTGGGG
CAGGTCCCCTTTGCCCTTCTCACTGGAACACGCGCACAGGGTGACGCACATGCCG
CACCCGCCCCTGCCTCCGATCCCGGCGCCCGTGGCGCTCCCCACCGTGCCTGGCA
GCGTGTGCGGCACGCTGGTCGACTCCCTTGGCCTACTGACGGAGGAGGAGCTGCT
GACTAACCAGCAGCTGGAGTTCACGTGGTCCCCGGGCAGTGCCGGTGCGCACAAG
CTGTTTGCCAAGTTTGTGTCCAGGCGTCTTCGCCAGTTCCAGCACGATCGGGCCA
AGTCAGACCGGAACAGCACCTCGCGCCTGTCGCCGCACGTGCACTTCGGCGAGAT
CAGCGTGCGGTACATGTACTACGTGGTCAAGCAGAAGGAAGCCGAGTGGCTGGCG
ACGGGAGAGCGCATCACCAGCTGTGCGGACTTCCTGCAGCAGATGGGGTACCGCG
AGTACTCGCGCTACCTCTCCTGGCACTTCCCCTTCACCCACGAGCGCTCCCTGCT
TGAGCACGTGCGCGCCTGCCCGTACCGCATGGACCAAAGGCTGTTCAAGGCTTGG
CGGCAGGGCAACACCGGGTACCCTCTGGTTGACGCGGCCATGCGAGAGCTGTGGA
GCTCCGGCTGGATGCACAACCGCATGAGGGTGACCGCTGCTTCCTTTTTGGTCAA
GCACCTGCTGCTGCCCTGGCAGTGGGGCCTGAAGCACTTTTGGGACGCGCTGGTG
GACGCAGATCTGGAGGCCGATGCACTGGGGTGGCAGTACTGCGCAGGCTGCCTGG
CAGACGCACACCCCCTGGACTACATGATCGACCACGGGACCGAGTCCAAGCGGTT
TGACCCCGACGGCCAGTACGTGCGCCGCTGGCTGCCCGTGCTGGCAAGGGTGCCC
AGGCAATACATACACGAGCCGTGGACCGCACCGCAGCATGTGCTGGACGAGGCGG
GCGTCGAGCTCGGCTCCAACTACCCCTGGCCGGTGGTCGACGCGGGGGAGGCGCA
GGTGCTGCTGGGCGCTGCCAAGGACGTGGTGGACGCCGCCCTGGAGGCGCTCAAC
GAGACGCAGAGAGGGCCCTACCGGCCGCCCAGCCTCAACCCCCTCGACCCATCCG
CGCCGGACTCGCTGCGGCGCATGTATGCCAAGTTCCAGCACCAGCTCACGCGCAC
GGCGGAGTCGGCGGCCAGCGGGCAGCGGCCCTCCAAGCGGACAACGATGGAGGCA
GCAGCGCAGCAGGCACGGGAGGACATGCAGGTGTGCGGCCAGGGCCAGGGCGGCT
CCTCTGACAACGAGGAAGTCGAGAGCAACATGGGGGTGGTTGACGCGCTGCTCGG
CGGCATGGCGCAACTCGCCGGTGATGACACGGCGGAGGTGGTGCAGCGCCGCCAC
GTGCACCGCCGCCGGCCGTCCTCCTGCGCAACAGGAGGCAGTACGCAGGACGGCC
CGCTCGCGCGCCGACCCGCCGCCGGCAGCGGATGCGGCAGCGCGCCAGATGCTCC
ACAGCAAGATGGGGCATCGCCAGACGCTCCTTGGCAACACGAGGCACCGCCCCGA
GGCAATGGCCAAGCAAAGCATGCGGCTCTGCAGCCAAGCAACGCATGGGGCTCTT
GA
SEQ ID NO: 11-DNA, artificial, sgRNA-1
TCTGGCGCCCATATGAACAGCGG
SEQ ID NO: 12-DNA, artificial, sgRNA-2
CCATGGCTCGTACAGCAGGTCGG
SEQ ID NO: 13-DNA, artificial, sgRNA-3
ACTGTACGATCCCATCACCATGG
SEQ ID NO: 14-DNA, Oocystis sp., YVTN (or WD40 repeat domain)
atggccgagc cggacgcgaa cggcgcgtcg gatggcaagc gcgcggagat ttacacgtac
gagttcccca acctcgttta ctccatgaac tggacggtaa gcaagcacag ttgtagcgca
cacagccttg ggtgctgctg cgtccttgct gatggtcatg cttgcgtcgg ccacccgttt
tctcacctac cgaaactggc tgcgcacccc tttcagttat gcttgtaatc accgcctcat
tacctttgtg tgcagtctcg tcgggacaag aagtttcgac tggcagtggg cagcttcatc
gaggactata ataacgtcgt caatatcata tcctgtacgc gccttccccg cactacccag
cgtagcggct cagtctgtcg tgcggggttg gcacgacagt cgttgggttg aagactattt
gaactattgc tgattagtga cgcccatgct ctctcgtaaa gcggggcgga tggctccttt
cgcgcccact accatctggc cgcgacctgt ggctctgacg cctcgctggg tctcgacctg
ctgaccatac tccctcgtta ctggctgcaa cgcagtggac gaggaacagg gcaagtttgt
atgcgacccg tcactgacct tcaagcatcc gtacccaccg accaaggtga tgtttgtgcc
agaccgggaa ggcactcggc ccgacctgtt ggccaccacc ggcgactatc tgcgcgtgtg
gaagatcggg gaggatggcg tcacgctgca gaagctgctg aacgatgtaa ggcctagatt
aacgtccagc gctgtgggga aggaccgacg gcacgggccg gaaagggaca tgcatgccgt
accgggggtg atcacgggcg acggcccacc ggatcatcat cttcctcgct tccaccaacc
ctgcgcaacg tccttcgcaa cgtcttgaac aatattttgc atctttcacg ttcatccatc 960
ctcgtcatgg cgaaattaac ttgcagaaca agaacagcga gttttgcgcg ccgctcacat 1020
cgttcgactg gaacgagacc gaccccaagc gcctgggcac cagctctatc gataccacgt 1080
gcacgatctg ggacatcgag aagggcgtgg tggacacgca gctcatcgcg cacgacaagg 1140
aggtgtatga catcgcgtgg ggcggcgtcg gcgtcttcgc gtcggtgtcc gccgacggct 1200
cggtgcgagt cftcgacttg cgagacaagg agcatagcac gatcatctac gagacgccgt 1260
cgccagagac gccgctgctg cgcctggggt ggaacaaaca ggaccccagg tacatggcga 1320
cgatcgtgat ggactctaac cgcgtggttg tgctggacat ccgcgtgccc accgtgcctg 1380
tcgccgagct gcagcggcac caggcgtgcg caaacgcgct tgcctgggcg ccgcacagca 1440
gctgccacat ctgcacagcg ggcgacgacg cacaggcgct gatctgggac ctcagcgcgg
tatccaagga gggcgactcg ggcctggacc ccatcctcgc gtacaatgca ggtcaagagg 1560
taaaccagct gcagtggtct tcgacgcagc ccgattgggt ggcagtctgc tttggcaaca 1620
aggcgcagat cctgcgagtg tga 1643
SEQ ID NO: 15-DNA, Oocystis sp., RNA binding domain
atggaggtcg ctacaaacgg cgaccacgcg cagcaccacc tcggcatgcc gcacggcgcg 60
cggtctgaat acagcagtgg cagcgatatg tcgcgcggcg gcggtcatgc gggcagcagc 120
ggcggccagg ctcagcagca gcaggctcag caggccgccg cagctggcga ggccggccca 180
ccgcgcaagc tggtgatcct tggcctgcca tattttacaa gcgacgacac tctgcatggc 240
tacttttctc agcttggtca ggtagaggag gcgctggtca tgcgtgatca cgcgtcgggt 300
cgctcccgtg ggttcggatt tgtgacgttt attaccgccg aagacgctgc gcgcgtggct 360
gggcgggagt acagcgtcga tggtcggcga tgcgaggcaa agttcgcgct gccgcgtggc 420
gagagcgcaa gccagcgtgt tacgcgcatc ttcgtggcaa agttgccacc gcacgttgcc 480
gaggacgagc tgcgcaccta ctttgagcag gtgcgacgcc ccctggggct gggttccaac 540
aactgcccta gcaccggtca caaggtgcct ttaggcttct gcaggaatgt gcctccggtt 600
aatgtattga ttcatcaacc ttatcagcgt attggtgctg cattcaagtg gtgatcacca 660
cgtgcatctg ccagctgtct ctcgatcacg tctccaaacg gcgatccgcc tagtccgccc 720
aagccctgcc caagcgcgtc gtcgtgctgg gccccccgtt ccgtgtgcaa gacggtgtgc 780
ttgcttacga ggtgtatacc gtttttattt tcttacgcgc agtacggagc gattcaggat 840
gtgtacatgc ccaaggatgc ttccaagcag gcgcgacgtg ggattgggtt cgtgacgttc 900
gcgagccccg aggcggttga cgccgtcatt cgcacatccc acgtgttaca cggccaggag 960
ctcgtcgtcg ataaggccgc gccaaagcag aaagagccgt ttccgcttgg agctggccta 1020
ccaggcgcca cagccagtgg cgtgccttac cggtcggcgc agccgtcgct gtcagccgaa 1080
cggcttgcca gcttgagcaa cggcgcattc gccggcttga ttcccggcgg ctacggcttc 1140
ggcggtcatg ggctgcagca gcagcagcac atactgccgg ggtcaatggc tggtggcgcg 1200
ctcagcggtt ctgcaggatc gctatacgac tcgttcggag gcgcgatcaa cggtactcaa 1260
gaagacggaa acggagacat gggcctttcc ggtaagcact tgctttttgc gtgcaatcta 1320
gataaaggga tatttacgtt catccatact tgtattcgca cccaggaaaa tccaggcagt 1380
tgcctgatct cacgtatttt tcgtgcacgc cacgccccaa gtggaaagga ctatggaatt 1440
attaaagagt ctgcgcagaa cagacgctag ttccttcggt tctgtcagac cctacacttg 1500
ctttgctgca cgccacgccc caatcaacgt atccccctcg gtgtgcactg cgctgcccgt 1560
acggcttgca aggatccaac ccaggcatgc agtatgctat tgcacagctg gcccgtgcgc 1680
agcaggcggc gcagcttggt acgtgccgcc gctgctgaac ctcaacccgc ctcccacttc 1740
atcacgctcc gttcttattg cttctttgtg cctagtatat cccatgcctc gatggcgtct 1800
cgcagtcaat aacccacaca ccaacacact aacgaattct gcatcacttg tgcggcaggt 1860
ctctcgctga catttgacgg tcgcccaagc agctccctca acctgggcgc agctgcgacg 1920
gccgcggccg cacagaatgg cggccggccg gcgggcgcgc cgggcagcgc caactcgctc 1980
acagacctcg accggatgta tggcgtgcag cagcagcagc aagcaggtgc gtgtggcgtt 2040
ctgcccagct tggctggaat tattgcgcga aaggacgttc tattgatgtt ctagacaaat 2100
gacctgggtt ggaactatct cgtttttttt ttaacgtgac caagaggcct gtcgtgccag 2160
gaattgctag cgagcatggt ttgagcacga gcgcgtacgc tcaccgcagt tcctcgctca 2220
gtccagccgc ctgggtttgt acgtcgcctc tgcatcgttt gagcgctgca tcctcctgct 2280
ccgttgctca ccccacctcg cccccaccca ccccctgcag ggctttccac tggcctgccc 2340
aacggcctgg tctcgctgcg cggcacaggc gcaggcggcc cgaccagccc cggtggtgga 2400
cgcggccctg gcggcattgg cgctgagccg gtcgccgcag gtggtaccag tgctggcgcc 2460
ggcggggcac tgtgcaccaa ccgcgtgttc attggcaagc tgggcaagga tgtgatggag 2520
gcggacatta aggagtactg ctcgcgattt gggtacgtgc tggacgtgta catcccgcgc 2580
gacaaaaaca acaagcgaga gcatcgcggc tttggctttg tgaccttcga gaccgaggcc 2640
gcggtcgatc gcatccttgc gtttgatgac caccaaatcc acggctcggt gattgccgtc 2700
gaccgagcgc tgccgaggca ggaggacacg agccagagca gcgtggcgct cagtggtgac 2760
cagcagtatg gcgctgacgt cagcagtgac gctgtcagcg ccgcactcgg gatggccgcg 2820
cttggcctgg gcgccaacgg acaggtgctg ccggggcctg cgcgccacaa caacgaccgc 2880
aaceggtate tgtaccagcc ctactag 2907
SEQ ID NO: 16: DNA, Oocystis sp., ADP-glucose pyrophosphorylase (APGL)
ATGCAGTCGCTGCACAGCCAGGCGCCCACCGTGGGCAACTGCGGGCAACTGTCGA
GGCAACAACGGCGAAATGCTGGCCTGGGATCGGCCTTCTCTGGCGGCCCCCTTGC
TGGCAAGGCGTTTCTAGCGGGCAGCAAGCTGACCGTTCGGGCAAGCAGCAAGTCT
GGCGTGAGGGGCGCGGGCAAGCTGCAGGTGCAAGCAGTTATCACCAAACAGAGTG
CTGATCAGGGTTATATGACCGAGAGCATCTCGACCAGGAGTGTGGCAGCGATCAT
CCTCGGCGGTGGCGCGGGCACTCGCCTGTACCCCCTGACCAAGCAGCGCGCGAAG
CCGGCCGTGCCTATTGGCGGAGTGTACCGCCTTATCGACGTGCCCATGTCCAACT
GCCTCAACTCGGGGATCAGCAAGATCTACATCCTGACGCAGTTCAACTCCACGTC
GCTGAACCGCCACCTGGGCCGCACCTACAACTTCGGCAACGGCCCTCGCAGCGGT
GGCGATGGCTTCGTTGAGGTGTTGGCAGCCACGCAGACTCCCACCGACGCAACCT
GGTTTCAGGGTACCGCCGATGCTGTGCGACAGTACACGTGGCTGCTGCAGGATAT
CAAGAACAGGACAGTGGAGGACATTGTGATTCTGTCGGGAGACCACCTGTACCGC
ATGGACTACATGAAATTCGTGAACCAGCACCGTGAGACCGGGTCAGACATCACCG
TCGGCTGCCTGCCTTGCGATCCCGAGCGTGCCGCGGACTTTGGACTCATGAAGAT
CGACAATGAGGGACGCATCGTTGAGTTTGCTGAGAAGCCCAAGGGTGACGCGCTG
CAGGCGATGAAGGTGGACAACACACTGCTGGGGCTGTCAAAGACGGAGGCTGCGG
AGAAGCCGTTCCTTGCGTCTATGGGCATCTACGTGTTCAAGAAGTCTGCGCTTAT
TGACCTGCTGACCAAGGAGTACCCCAACGACAACGACTTTGGCGGCGAGATCATC
CCCAAGGCTGCGGCAGGAGACTACAAGGTGTCTGCCTACCTGTTCAACGGCTACT
GGGAGGACATCGGGACCATCAAGTCGTTCTTCGACGCAAACCTGGCGCTGGCGCA
GCACCCGCCGCGCTTTGAGTTCTACGACCCGCAGCAGCCCATCTACACCAGCCCG
CGCTTCCTGCCGCCCGCAAAGATAGTGCGGTGCAAGGTGCAGGACGCGATGGTCT
CGCCCGGGTGCTACCTGGCGGACTGCACGGTGGAGAATGCCATCATCGGGCTGCG
CAGCCGCATCGAGAAGGGCGCCGTCATTCGGGACGCCATGGTGATGGGCGCGGAC
TACTACGAGAACGATGCGCAGCGCGCCGCACTCAACGCGGCGGGCCGCGTGCCCA
TCGGCGTGGGTGAAAACAGCACCATAATGAACGCCATCCTGGACAAGAACGCGCG
CATTGGCAAGAACTGCTCCATCGTGAACAAGGACGGCGTCGACGAGGCCAGCCGG
GAGGAGGACGGCTTCTTCATCCGCAGTGGCATCATCACTGTCTGCCGCAACGCCG
AGATTCTGGACGGCACAGTTGTCTAA
SEQ ID NO: 17: DNA, Oocystis sp., protein kinase (or PK1)
ATGCGCGATCCCAGTGGGAGTGCCACGCGACCTACTCCTGCTCGGGTTCACCTGG
CGGGGACCCCAGCCCATGCAAACCAATGCCCCAGGATTTCAGCGTGTCTGATCAG
CAGGTAACGGCGCCTTTGACGCGCCTGATGCCGCTGGAGCTGCGTGTACGACATT
GCCGCACGATCTAGTGGCGCTCACGACTGCAGGGACATGGTAGCCCAGGAACACG
GAGACGGGCCGCCCAGGTGGCACAGCCATCCCAGACATCGGCTGTGTCCAGTAGT
TCGTGTGTATCTTCGCCACGTATCAACGGTGCGGTGGAGGCGTTGGACCGACACA
AGCCAGCGAGCAACGCACGCGTTGCATCAGCTGCAGCAGGCCAATCAAGCACCGG
AGCAGCGGCACAGGCACAGCGCAATTTGCTCACGAAGCCTTTCAAGGGTCTGCAT
AACGACGGTCATACAACGAGAACTGGGACTTGATCATATGTGTCGGGGACGAGTT
TGTGTCCAGCTCCAACCTCAAGTACGTCGTCATCGACTTGCTGGGCCAGGGCACG
TTCGGCCAGGTAGTGCGCTGCTGGTGCGACCAGACGCAGGAGTATGTAGCCGTCA
AGGTGATAAAAAATCAGCCGGCTTACTATCAGCAAGCGCGCGTAGAGGTTGGCCT
GTTGCAATACCTCAACCGCTGTGCGGACGCGGACGACGTCCGGCACATTGTGCGC
CTCCGCGACTACTTTTTGTTCCGTAACCACTTGTGCCTCGCGTTCGAGCTGCTGT
CGGTCAACCTGTACGAGCTCATCAAGCACAACCAGTTCCGTGGGCTGTCTGCGGG
GCTCGTGCGCGTGTTCATCGCTCAGCTGCTTGATGCGCTGGTGGTGCTGCGTGAG
TCTCGCCTCATCCACTGCGACCTCAAGCCGGAGAACGTGCTGCTTACGGGCGCTG
AGTCAGCTGACATAAAGGTCATCGACTTTGGGTCCGCTTGCCTGGAGAGCAAAAC
GGTGTACAGCTACATCCAGAGCCGCTTCTATCGCTCTCCAGAGGTGGTGCTTGGC
TACCCGTACAACGTGGCGATCGACATGTGGTCCCTGGGCTGCATGGCCGCGGAGC
TGTTCCTTGGCCTGCCGCTGTTCCCGGGCGCATCGGAGCACGATTTGTTGTCTCG
GGTGGTGCAGGCGGTGGGCCTCCCGCCGCTGTACCTGCTGCAGGGCGCAAAGCAC
ACCAACAAGTATTTCAAGATGGTGGAGCGCGTGGTGCGGCTGCCAAGCGGCAGGT
CTGAGGTGGTGCCCGAGTATGTGATGCGCACTGCCGCGGAGTTTGAGGCTCTCAC
GGGGCTCAAGGCCACCACCGGCAAGCGCTACTTCTCACATACGCGCCTGCAGGAC
ATCATCAACTCATACCCTTCAGAGGGCGCGGGCAGCGAGCTGCGCCGCTCCCTTC
TGGACTTCCTTCGGGGCGTGCTGGACCCCGACCCAGCGGCGCGCTGGACGCCGCA
GCAGGCGGCGCGCCACCCGTTTGTGACGGGCCAGCCGTTCGCGGCGCCGTTCCAG
CCAGACGGGGAGCGTACGCCCCCGCCTCCCGGCTGGGACGCCTCCGCGGCCGGCA
TGCTGTCCAGCAGTGCTGGGCACCAGGCCGCTCAGCAGCAGCAGGCAGGGTGGAA
CGGCGCGTCCGCGTCGCCGCACTATGGCAGCGCGGCGGCGGCGATGCTCGCCACG
TCGCCGCACGTGCAGGCGCATGTGGCGGCCATGGCGGCGCTCGCCCAGCAGCAGC
AGCAGCAGGGCATGGGCACCCCGTCAGGGCTGCCCTCGTACCGCCCGCAGCCTGT
GCCTGTACCGCGTGCGGGGCCCGGCTACGGCGGTGGCGGCGGCTGGGCAGCGCCG
TATCCCCACCAGCACCAGCAGCAGCAGCAGGCCGGCAGCCTTACGCAGCAGTACC
TGCAGCAGCAGCAGCAGCAGGCAGGCGGCAGCCTGGGCCTGTTCAGCCCGCCCGG
CCTGGGCCCCGCCAGCCTGATGCAGCTCGACCACCTGAGCGCGGCCTCGGGCTCG
TTCTTCAGCCCGCCTGGGTCGCTGGTCAACGCGACCGGGCGGCTGGCGCCAGCGC
CGGGGGCCGCGCGCTACGGCAGCTACCAGCCGATGCCGGTGGACATGGGCTTCAG
CCCCGCCGGCAGCCTGTCGGCAAACTCGGTGCTGGCGGTGGCCATCGCGGCAGCC
AACGCCGCAGCCGCCTCGGCCGCTGCGCAGCAGCAGCAGCAGCAGCACGGCGTCG
CGGAGCAGCTGCGGTTGCAGTGGCAGGGCGCCGCCGTGGGCGGCGGCGGCGGCGG
CGGCTTCCCCAGTGGGCTGGCGCGCATGTCGGGCGTGGCAGGGGGCTCGTACTCC
GGCCAGTCTGCAGTGCCGATGGTGGGGTCGTATGCCAGCAGCCTGCCGGGGGGCA
GCGCCGCCACCGCGGCGGCGGGCCCAGGCACGGGCTCAAACCCGCTGGCCATGCT
CGCGGCGCAGCAGGCGGCCGAGGACGCACTCGCCAGCAGCCTGCGGCGCGGGTCG
ATGGGCGCAGGCGCGAGCGGCGGGTATGGCGCCGGCTTCGGCGGCTCCGCAGCGG
CCACGGCCGCTCCCGCCGCCGCCGCGGCGGCGGCCCAGCAGCCTGCGCCGGTGGA
CATGTCGGCTGCTGCAGGCAGCCTCGGCGCGGTGCTGCAGCAGCTGCAGGCGACA
CAGGCCGCGGCGCAGGCGCAACAGTTTGTGGCGGCGGCCGCCGGTGGCGGGAGCC
GCGGCCGTACGCCGCCGCCGCCGGCGGACGGTGCGCTTGCCACCGCTTGCGCTGC
AGCTGCTGCGGGAGCAGTGGCCGGGGGGGGCGGCAGCGCCCACCAGCAGCAGCAG
CGGCACGAGTCGGTGTCGCCATCTCCTGGGGACTGGGACCCGCTGTACAGCGACG
ACCAGCTCCTGGAGGACGATACGCCCGCTCAGCAGCGTACACCGCCGCAACAACC
GCAGCAGCGCAAGGGCAGCGGCAACTTGGCAGCGGCGCTGGCAGCGGCCGTCACG
CTGCCTGCCCAGCAGCAGCAGTGGCAGCAGCAGCAGCAGCAGCGGCAGCCCCTCG
CGGGCGGGCTGTCTCCGCCAGACCCTGCGCTAACAGCGGCAGCCGCGGCGGCACT
TGGCGCGCTGCAGCAGCAGCAGCAGCAGCAGCAGGCCACGGCAGGTGCCGGTGCG
GCCACACTGGACACCGCAGACCTAGTGGGCTGGCTGCAGCAGCAGCAGCTGCTGC
GCGCGCTGCGCCCGCCTCCGGCAACGGCAGACGCCCGCGGCGCCGGAGGTGCCGC
CGCTGCCGCGGCCGCCTCCATTGAGCCTGCTGGTGTGGCGGGTGCCGTGTCGGCG
GCGGCGCCGGTGCCCGGCGACCAACAGCACCGTGCAGCAGGGCCAATGCCGCCCC
TCTCTGCTGCCGAGCAGCAGCAAACACAGGCCGGTAGCAGCGGGGGCGCTGCTGC
CCATGCTGCGGCAGCAGCAGGGGAAGGTGTTGCCGCCGGCCTGGGCGGCGGTGCT
GGTGGCGCCTCTCGGCGGAACAGTTTTGAGATTGATCGGAGGGGTAGTTTTGAGA
TTGGCTTTGAGGTTGCTCCAGTCGTGTCCACAGGCCTGCAGCAGGCGTCGGGAGC
GCGCACTGGCGCGGCACCCGCCCACGCCGGGGGTGGATTCAGCTGCTTGTCTGCG
CAGCTCCACGGCAGCAGGCAGCACAAGCAGCAGCAGCCGGAAGCGGCAGAACATT
CCAGCAAGTAG

What is claimed:

1. A recombinant photosynthetic organism comprising a deletion, disruption, or inactivation of:
a gene encoding a cryptochrome/photolyase FAD-binding domain comprising a nucleic acid sequence having at least 80% sequence identity to SEQ ID NO: 2; or
a gene encoding a cryptochrome/photolyase FAD-binding domain having at least 80% sequence identity to the polypeptide sequence of SEQ ID NO: 1;
wherein the recombinant photosynthetic organism exhibits higher biomass productivity and higher lipid productivity versus a corresponding control organism not having the deletion, disruption, or inactivation.

2. The recombinant photosynthetic organism of claim 1 wherein the gene encoding the cryptochrome/photolyase FAD-binding domain encodes a polypeptide sequence having at least 90% sequence identity to the polypeptide of SEQ ID NO: 1.

3. The recombinant photosynthetic organism of claim 1 wherein the gene encoding the cryptochrome/photolyase FAD-binding domain encodes a polypeptide sequence having at least 95% sequence identity to the polypeptide of SEQ ID NO: 1.

4. The recombinant photosynthetic organism of any one of claim 1 wherein the organism is a Chlorophyte alga.

5. The recombinant photosynthetic organism of claim 4 wherein the organism is of the Class Trebouxiophyceae.

6. The recombinant photosynthetic organism of claim 1 wherein the deletion, disruption, or inactivation is to a regulatory sequence of the gene encoding the cryptochrome/photolyase FAD-binding domain.

7. The recombinant photosynthetic organism of claim 6 wherein the regulatory sequence is a promoter.

8. The recombinant photosynthetic organism of claim 1 wherein the deletion, disruption, or inactivation comprises a deletion of one or more amino acids of the encoded cryptochrome/photolyase FAD-binding domain.

9. The recombinant photosynthetic organism of claim 1 wherein the deletion, disruption, or inactivation comprises a disruption by an insertion in the gene encoding the cryptochrome/photolyase FAD-binding domain.

10. The recombinant photosynthetic organism of claim 9 wherein the insertion comprises insertion of a stop codon in a sequence encoding the cryptochrome/photolyase FAD-binding domain.

11. The recombinant photosynthetic organism of claim 1 wherein the organism has at least 20% higher lipid productivity versus a control photosynthetic organism.

12. The recombinant photosynthetic organism of claim 11 wherein the organism has at least 30% higher lipid productivity versus a control organism.

13. The recombinant photosynthetic organism of claim 1 wherein the organism has at least 35% higher biomass productivity per unit time versus the corresponding control organism.

14. The recombinant photosynthetic organism of claim 1 wherein the recombinant organism has a FAME/TOC ratio of at least 0.4 after two days of cultivation.

15. The recombinant photosynthetic organism of claim 1 wherein the recombinant organism has higher biomass productivity under nitrogen deficient conditions.

16. The recombinant photosynthetic organism of claim 1 wherein the recombinant organism has higher total organic carbon production under nitrogen deficient conditions.

17. The recombinant photosynthetic organism of claim 1 from a family selected from the group consisting of: Oocystaceae, Chlorellaceae, and Eustigmatophyceae.

18. The recombinant photosynthetic organism of claim 17 wherein the recombinant organism is an alga of a genus selected from the group consisting of: Chlorella, Parachlorella, Picochlorum, Tetraselmis, and Oocystis.

19. The recombinant photosynthetic organism of claim 18 wherein the recombinant photosynthetic organism is an alga is from the genus Oocystis.

20. The recombinant photosynthetic organism of claim 1 wherein the gene encoding the cryptochrome/photolyase FAD-binding domain has a nucleic acid sequence having at least 90% sequence identity to any one of SEQ ID NO: 2, 4, 6, 8, or 10.

21. A biomass product comprising the photosynthetic organism of claim 1.

22. A recombinant photosynthetic organism comprising a genetic modification to:
a gene encoding a cryptochrome/photolyase FAD-binding domain that has a polypeptide sequence having at least 90% sequence identity to SEQ ID NO: 1; or
a gene encoding a cryptochrome/photolyase FAD-binding domain having a nucleic acid sequence having at least 90% sequence identity to SEQ ID NO: 2; and
wherein the recombinant photosynthetic organism exhibits higher biomass productivity and higher lipid productivity versus a corresponding control photosynthetic organism not having the genetic modification.

23. The recombinant photosynthetic organism of claim 22 wherein the genetic modification is a deletion, disruption, or inactivation.

24. The recombinant photosynthetic organism of claim 22 wherein the recombinant alga has at least 25% higher lipid productivity versus a control algae.

25. The recombinant photosynthetic organism of claim 22 wherein the recombinant alga has at least 35% higher biomass productivity per unit time versus the corresponding control photosynthetic cell or organism.

26. A method of producing a composition containing lipids comprising performing a genetic modification in a photosynthetic organism to:
a gene encoding a cryptochrome/photolyase FAD-binding domain having a polypeptide sequence having at least 80% sequence identity to SEQ ID NO: 1; or
a gene encoding a cryptochrome/photolyase FAD-binding domain having a nucleic acid sequence having at least 80% sequence identity to SEQ ID NO: 2;
wherein the recombinant photosynthetic organism exhibits higher biomass productivity and higher lipid productivity versus a corresponding control photosynthetic organism not having the genetic modification; and
cultivating the organism, and thereby producing a composition containing lipids.
